(19) United States
(12) Patent Application Publication
(10) Pub. No.: US 2021/0332112 A1
(43) Pub. Date: Oct. 28, 2021

(54) STABILIZED PERTUSSIS ANTIBODIES WITH EXTENDED HALF-LIFE

(71) Applicants: BOARD OF REGENTS, THE UNIVERSITY OF TEXAS SYSTEM, Austin, TX (US); SYNTHETIC BIOLOGICS, INC., Rockville, MD (US)

(72) Inventors: Jennifer MAYNARD, Austin, TX (US); Annalee NGUYEN, Austin, TX (US); Michael KALEKO, Rockville, MD (US)

(21) Appl. No.: 16/325,710

(22) PCT Filed: Aug. 15, 2017
(86) PCT No.: PCT/US2017/046917

§ 371 Date: Feb. 14, 2019

Related U.S. Application Data
(60) Provisional application No. 62/375,347, filed on Aug. 15, 2016.

(51) Int. Cl. C07K 16/12; A61K 45/06; A61K 39/40; A61P 31/04

(52) U.S. Cl. CPC C07K 16/1225; A61K 45/06; A61K 39/40; A61P 31/04; A61K 2039/505; C07K 2317/92; C07K 2317/94; C07K 2317/524; C07K 2317/72; C07K 2317/24

(57) ABSTRACT

The present invention relates, in part, to modified humanized antibodies which bind the pertussis toxin protein and their use as therapeutic agents. In particular, the present invention is directed, in part, to improved humanized 1B7 and 11E6 antibodies with extended in vivo half-lives.

This application claims the benefit of U.S. Provisional Patent Application No. 62/375,347, filed Aug. 15, 2016, the entirety of which is incorporated herein by reference.

### FIELD OF THE INVENTION

The present invention relates, in part, to humanized antibodies derived from murine antibodies 1B7 and 11E6 which bind the pertussis toxin protein. In particular, inter alia, the present invention provides improved humanized 1B7 and 11E6 antibodies with extended in vivo half-lives.

### BACKGROUND

Pertussis, or whooping cough, is an acute infectious disease caused by the bacterium Bordetella pertussis. Pertussis is associated with uncontrollable, violent coughing and causes an estimated 300,000 deaths each year worldwide. Infants with pertussis often require hospitalization in pediatric intensive care units, and their treatments involve mechanical ventilation. Pertussis in adults generally leads to a chronic cough referred to as the “cough of 100 days.” The incidence of pertussis is increasing due to exposures of unvaccinated and under-vaccinated individuals including infants who are not yet fully vaccinated, individuals whose immunity has diminished over time, and asymptomatic carriers.

With no specific therapeutics to treat disease, pertussis continues to cause considerable infant morbidity and mortality. While antibiotic treatment can eliminate the B. pertussis bacteria from the respiratory tract, it does not neutralize the pertussis toxin protein, a major contributor to disease, responsible for local and systemic effects including leukocytosis and immunosuppression, and thus has minimal effects on the course of pertussis. In contrast, antibody therapy offers the advantage of specificity that antibiotic treatments may lack. Antibodies that bind the pertussis toxin protein have been developed. However, the effectiveness of these antibodies in patients is either minimal or unclear. As such, methods for enhancing the efficacy of antibody-based therapies against the pertussis toxin proteins can be clinically beneficial.

There remains a need for improved antibody-based agents against the pertussis toxin protein with increased efficacy and reduced side effects.

### SUMMARY

In various aspects, the present invention is directed to one or more modified humanized antibodies that bind to and/or neutralize a pertussis toxin protein. In some embodiments, the present modified humanized antibodies are modified to provide increased stabilization and extended in vivo half-lives. In some embodiments, the modified humanized antibodies have the advantage that smaller dosages and/or less frequent dosing are required in the therapeutic use of the antibodies.

In various embodiments, the modified humanized antibodies of the invention comprises an IgG constant region that has one or more amino acid modifications which increase the affinity of the constant region or fragments thereof for the neonatal Fc receptor (FcRn). In some embodiments, the constant region is derived from IgG1. In some embodiments, the constant region is derived from human IgG1. In some embodiments, the modified humanized antibodies of the invention comprise an FcRn binding fragment (e.g., Fc or hinge-Fc domain) of an IgG constant region. In various embodiments, the IgG constant region comprises one or more mutations at amino acid residue 252, 254, 256, 433, 434, or 436 (residues as depicted in FIG. 1 or analogous residues of other IgG molecules as determined by sequence alignment). As used herein, all residues of the IgG constant region are numbered according to Kabat et al. (Sequences of Proteins of Immunological Interest, U.S. Department of Health and Human Services, 1991, which is incorporated by reference herein in its entirety). In an embodiment, the IgG constant region includes a triple M252Y/S254T/T256E mutation or YTE mutation.

In various embodiments, present modified humanized antibody that binds a pertussis toxin protein further includes an immunoglobulin heavy chain variable region and an immunoglobulin light chain variable region.

In various embodiments, the modified humanized antibody is a 1B7 antibody that includes an immunoglobulin heavy chain variable region comprising a CDR_{H1} comprising an amino acid sequence selected from SEQ ID NO:26, a CDR_{H2} comprising an amino acid sequence selected from SEQ ID NO:27 and SEQ ID NO:28, and/or a CDR_{H3} comprising an amino acid sequence selected from SEQ ID NO:29; and/or an immunoglobulin light chain variable region comprising a CDR_{L1} comprising an amino acid sequence selected from SEQ ID NO:30, SEQ ID NO:31, and SEQ ID NO:32, a CDR_{L2} comprising an amino acid sequence selected from SEQ ID NO:33 and SEQ ID NO:34, and/or a CDR_{L3} comprising an amino acid sequence selected from SEQ ID NO:35. In various embodiments, the modified humanized antibody comprises 1, or 2, or 3 of CDR_{H1}, CDR_{H2}, and CDR_{H3} as described above. In various embodiments, the modified humanized antibody comprises 1, or 2, or 3 of CDR_{L1}, CDR_{L2}, and CDR_{L3} as described above.

In some embodiments, the modified humanized antibody is an 1B7 antibody that includes an immunoglobulin heavy chain variable region comprising an amino acid sequence selected from SEQ ID NO:1, SEQ ID NO:2, SEQ ID NO:3, SEQ ID NO:4, SEQ ID NO:5, and SEQ ID NO:6, and an immunoglobulin light chain variable region comprising an amino acid sequence selected from SEQ ID NO:7, SEQ ID NO:8, SEQ ID NO:9, SEQ ID NO:10, SEQ ID NO:11, and SEQ ID NO:12, or variants thereof.

In various embodiments, the modified humanized antibody is an 11E6 antibody that includes an immunoglobulin heavy chain variable region comprising a CDR_{H1} comprising an amino acid sequence selected from SEQ ID NO:36, a CDR_{H2} comprising an amino acid sequence selected from SEQ ID NO:37 and SEQ ID NO:38, and/or a CDR_{H3} comprising an amino acid sequence selected from SEQ ID NO:39; and/or an immunoglobulin light chain variable region comprising a CDR_{L1} comprising an amino acid sequence selected from SEQ ID NO:40, a CDR_{L2} comprising an amino acid sequence selected from SEQ ID NO:41, and/or a CDR_{L3} comprising an amino acid sequence selected from SEQ ID NO:42. In various embodiments, the modified humanized antibody comprises 1, or 2, or 3 of CDR_{H1}, CDR_{H2}, and CDR_{H3} as described above. In various embodiments, the modified humanized antibody comprises 1, or 2, or 3 of CDR_{L1}, CDR_{L2}, and CDR_{L3} as described above.

In various embodiments, present modified humanized antibody is an 11E6 antibody that further includes an immunoglobulin heavy chain variable region comprising an amino acid sequence selected from SEQ ID NO:13, SEQ ID NO:14, SEQ ID NO:15, SEQ ID NO:16, SEQ ID NO:17, and SEQ ID NO:18, and an immunoglobulin light chain variable region comprising an amino acid sequence selected from SEQ ID NO:19, SEQ ID NO:20, SEQ ID NO:21, SEQ ID NO:22, SEQ ID NO:23, and SEQ ID NO:24, or variants thereof.

In various embodiments, the present modified humanized 1B7 and 11E6 antibodies show improved specificity. In an embodiment, the modified humanized 1B7 antibody binds the pertussis toxin protein with a K_{D} of less than about 3 nM, or about 2 nM, or about 1 nM, or about 0.5 nM. In another embodiment, the modified humanized 11E6 antibody binds the pertussis toxin protein with a K_{D} of less than about 12 nM, or about 10 nM, or about 8 nM, or about 6 nM, or 4 nM, or 2 nM, or about 1 nM, or about 0.5 nM. Further, the present modifications allow for an extended in vivo half-life in addition to this improved specificity.

In various embodiments, the present invention also provides nucleic acids, expression vectors, host cells, and methods for making the modified humanized 1B7 and 11E6 antibodies. The present invention also provides pharmaceutical compositions comprising the modified humanized 1B7 and/or 11E6 antibodies.

In one aspect, the method of the invention involves treating a patient with Bordetella pertussis, comprising administering to the patient the present modified humanized 1B7 antibody and/or the modified humanized 11E6 antibody, or pharmaceutical compositions including the antibody or antibodies. In an embodiment, the modified humanized 1B7 antibody and the modified humanized 11E6 antibody are co-administered to the patient producing synergistic effects. In another embodiment, the method includes administering to the patient the modified humanized 1B7 antibody and/or the modified humanized 11E6 antibody, along with antimicrobial agents.

In a further embodiment, the method of the invention is directed to preventing Bordetella pertussis infection in a subject by administering to the subject the modified humanized 1B7 antibody and/or the modified humanized 11E6 antibody, or pharmaceutical compositions including the antibody or antibodies.

In various embodiments, the methods of treatment or prevention relate to infants.

In some embodiments, the method of the invention involves preventing the onset of pertussis by prophylactically administering the modified humanized 1B7 antibody and/or the modified humanized 11E6 antibody, or pharmaceutical compositions including the antibody or antibodies, to a subject, including an infant that has yet to be vaccinated (e.g. such as, for instance, whole-cell vaccines and acellular vaccines, inclusive of the vaccine in DTaP, and the like).

In one embodiment, the method of the invention comprises reducing white blood cell count in the patient. In another embodiment, the method of the invention comprises reducing the duration and/or the frequency of cough in the patient. In a further embodiment, the method of the invention comprises reducing the levels of the Bordetella pertussis in the nasopharynx and the lung of the patient. In another embodiment, the method of the invention neutralizes the pertussis toxin protein.

In another aspect, the method of the invention involves treating a patient with Bordetella parapertussis, comprising administering to the patient the modified humanized 1B7 antibody and/or the modified humanized 11E6 antibody, or pharmaceutical compositions including the antibody or antibodies. In another aspect, the method of the invention is directed to preventing Bordetella parapertussis infection in a subject by administering to the subject the modified humanized 1B7 antibody and/or the modified humanized 11E6 antibody, or pharmaceutical compositions including the antibody or antibodies.

Other aspects and embodiments of the invention will be apparent from the following detailed description and examples.

### DESCRIPTION OF THE DRAWINGS

FIG. 1 shows the amino acid sequence of the human IgG1 hinge-Fc region (SEQ ID NO: 25) containing a hinge region, CH2 domain, and CH3 domain. Kabat numbering is as shown.

FIG. 2A-C, panel A shows SDS PAGE analyses of hu1B7 and hu1B7-YTE antibodies. Antibodies were generated by transient transfection in CHO cells and subjected to reducing and non-reducing SDS PAGE analyses. Panel B shows comparison of hu1B7 and hu1B7-YTE antibodies by size exclusion chromatography. Panel C shows Hu1B7 and hu1B7-YTE antibodies bound pertussis toxin with similar affinities.

FIG. 3 shows B. pertussis titers in nasopharyngeal washes (NPW). Nasopharyngeal washes obtained from control, hu1B7-treated and hu1B7-YTE-treated animals were collected on day 3 or 4 after B. pertussis infection. B. pertussis titers were obtained from quantitation of bacterial colonies following plating of dilutions of the nasopharyngeal washes. All animals were similarly and heavily colonized with no significant differences between the groups (ANOVA with unequal means). Data is shown for 6 control, 7 hu1B7-treated and 4 hu1B7-YTE treated animals.

FIG. 4A-B, panel A shows hu1B7 elimination kinetics in hu1B7-treated neonatal baboons. The half-life of the hu1B7 antibody was calculated for each of the hu1B7-treated baboons. The average elimination half-life was 11.8±4 days (with a range of 7-14 days). The average amount of hu1B7 antibody in treated animals at the time of infection was 35±18 μg/ml. Panel B shows hu1B7-YTE elimination kinetics in hu1B7-YTE-treated neonatal baboons (n=4). The half-life of the hu1B7-YTE antibody was calculated for each of the hu1B7-YTE-treated baboons. The average elimination half-life was 23±64 days (with a range of 19-32 days). The average amount of hu1B7 antibody in treated animals at the time of infection was 84±25 μg/ml.

FIG. 5, graph shows survival data for hu1B7-treated neonatal baboons vs. control animals. 3 of 6 controls were euthanized (the model is at LD₅₀), whereas 0 of 7 hu1B7-treated and 0 or 4 hu1B7-YTE-treated animals were euthanized (log-rank test gives p=0.0402). These data show that treatment with either hu1B7 or hu1B7-YTE improved survival in the primate model.

FIG. 6, graph shows maximum leukocytosis in B. pertussis-infected newborn baboons measured as CD45+ white blood cell count). Peak white blood cell counts from control, hu1B7-treated and hu1B7-YTE-treated animals are displayed. The white blood cell counts were significantly increased in the control animals compared to hu1B7-treated or hu1B7-YTE-treated animals (p<0.01). No significant difference between the hu1B7-treated and hu1B7-YTE-treated animals was observed.

FIG. 7A-B, panel A, graph shows the CD 45⁺ white blood cell count (WBC) at the indicated time points for control (dashed lines), hu1B7-treated (solid lines, black icons) and hu1B7-YTE-treated (solid lines, gray icons) animals. Control animals have rapid onset of leukocytosis. Both treated groups of animals exhibit a smaller and delayed increase in WBC. Panel B, graph shows the CD45⁺ white blood cell count (WBC) at indicated time points for hu1B7-treated animals (dashed lines, square icons) and hu1B7-YTE-treated animals (solid lines, round icons). These data show that hu1B7-YTE treatment suppresses leukocytosis (n=4).

### DETAILED DESCRIPTION

The present invention is based, in part, on the discovery of modified humanized 1B7 and 11E6 antibodies that exhibit improved biological activities. Because of the binding and/or neutralizing activity of these antibodies against the pertussis toxin protein, they are useful for treating patients infected with the Bordetella pertussis bacteria. The disclosed antibodies are engineered to target the pertussis toxin protein with high specificity while causing minimal side effects in patients. Furthermore, the disclosed antibodies exhibit enhanced stability and long in vivo half-lives. Various features and aspects of the invention are discussed in more detail below.

As used herein, unless otherwise indicated, the term “antibody” means an intact antibody (e.g., an intact monoclonal antibody) or antigen-binding fragment of an antibody (e.g., an antigen-binding fragment of a monoclonal antibody), including an intact antibody or antigen-binding fragment that has been modified, engineered or chemically conjugated, or that is a human antibody. Examples of antibodies that have been modified or engineered are chimeric antibodies, humanized antibodies, and multispecific antibodies (e.g., bispecific antibodies). Examples of antigen-binding fragments include Fab, Fab′, F(ab′)2, Fv, single chain antibodies (e.g., scFv), minibodies and diabodies. An antibody conjugated to a toxin moiety is an example of a chemically conjugated antibody.

Stabilized Antibodies that Bind the Pertussis Toxin Protein

In various aspects, the present invention is directed to one or more modified humanized antibodies that bind to and/or neutralize a pertussis toxin protein. In some embodiments, the present modified humanized antibodies are stabilized and have extended in vivo half-lives.

In various embodiments, the modified humanized antibodies of the invention comprise an IgG constant region. As used herein, the term “constant region” refers to the portion of an immunoglobulin molecule having a more conserved amino acid sequence relative to the other portion of the immunoglobulin, the variable region, which contains the antigen binding site. The constant region contains the CH1, CH2 and CH3 domains of the heavy chain and the CHL domain of the light chain.

In various embodiments, the IgG constant region is derived from the IgG1 subclass of IgGs, but may also be from any other IgG subclasses of given animals. For example, in humans, the IgG class includes IgG1, IgG2, IgG3, and IgG4; and mouse IgG includes IgG1, IgG2a, IgG2b, IgG2c and IgG3. It is known that certain IgG subclasses, for example, mouse IgG2b and IgG2c, have higher clearance rates than, for example, IgG1 (Medesan et al., Eur. J. Immunol., 28:2092-2100, 1998). Thus, when using IgG subclasses other than IgG1, it may be advantageous to substitute one or more of the residues, particularly in the CH2 and CH3 domains, that differ from the IgG1 sequence with those of IgG1, thereby increasing the in vivo half-life of the other types of IgG. In various embodiments, the IgG may be from any animal origin including birds and mammals. In various embodiments, the IgG may be derived from human, rodent (e.g., mouse and rat), donkey, sheep, rabbit, goat, guinea pig, camel, horse, or chicken. In some embodiments, the constant region is derived from human IgG1.

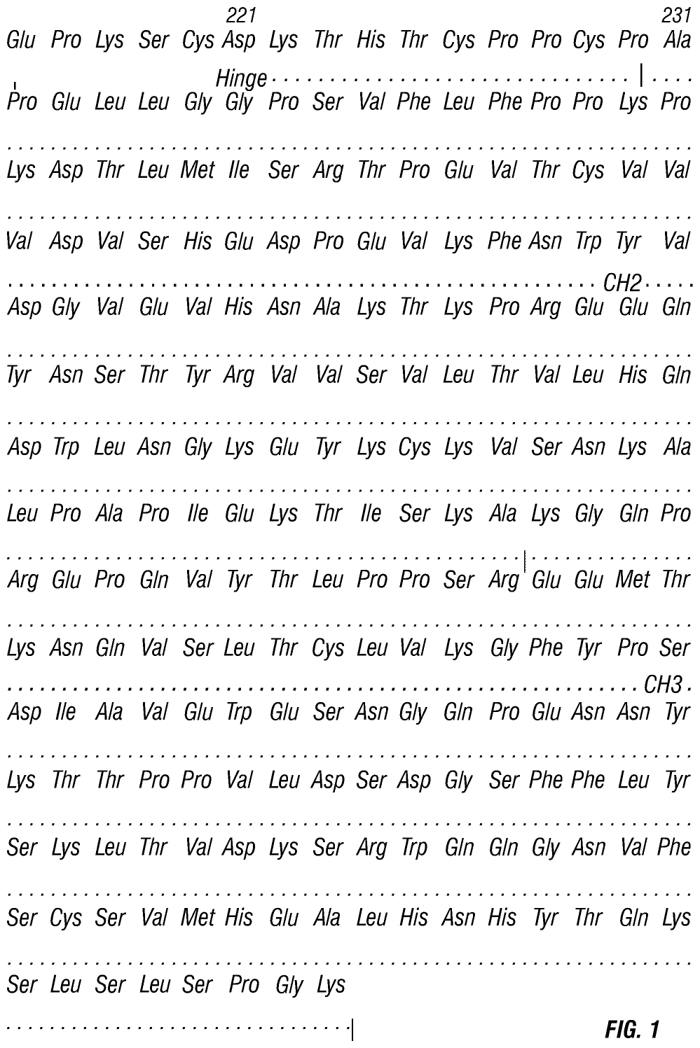

In various embodiments, the modified humanized antibodies of the invention comprise an FcRn binding fragment (e.g., Fc or hinge-Fc domain) of an IgG constant region. As used herein, the IgG Fc region refers to the portion of an IgG molecule that correlates to a crystallizable fragment obtained by papain digestion of an IgG molecule. The Fc region includes the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (e.g., residues 231-340 of human IgG1 depicted in FIG. 1, according to the Kabat numbering system) and the third constant domain CH3 (e.g., residues 341-447 of human IgG1 depicted in FIG. 1). The hinge-Fc fragment, as used herein, refers to a region of an IgG molecule including the Fc region (residues 231-447) and a hinge region (residues 216-230 of human IgG1 depicted in FIG. 1) extending from the N-terminus of the Fc region.

The present invention utilizes IgG constant regions that naturally contain an FcRn binding domain. However, in alternative embodiments, constant regions derived from other non-IgG immunoglobulins (e.g., IgE, IgM, IgD, IgA and IgY) or fragments may be utilized. In such embodiments, the non-IgG constant region or fragments thereof may be engineered to contain an FcRn-binding fragment. In such embodiments, the FcRn-binding domain comprises one or more amino acid modifications that increase the affinity of the constant region fragment for FcRn.

In various embodiments, the modified humanized antibodies of the invention comprise an IgG constant region that contains one or more amino acid modifications relative to a wild type IgG constant region. In various embodiments, the modifications increase the affinity of the IgG constant region for the FcRn. In some embodiments, the IgG constant region comprises one or more mutations at amino acid residues 251-256, 285-290, 308-314, 385-389 and 428-436, or equivalents thereof. As used herein, all residues of the IgG constant region are numbered according to Kabat et al. (Sequences of Proteins of Immunological Interest, U.S. Department of Health and Human Services, 1991, which is incorporated by reference herein in its entirety) and as presented in FIG. 1 (SEQ ID NO:25), and include corresponding residues in other IgG constant regions as determined by sequence alignment.

In some embodiments, the modified humanized antibodies of the invention comprise an IgG constant region that contains one or more amino acid substitutions at amino acid residue 252, 254, 256, 309, 311, 433 or 434, or equivalents thereof. In an embodiment, the amino acid substitution at amino acid residue 252 is a substitution with tyrosine, phenylalanine, tryptophan or threonine. In an embodiment, the amino acid substitution at amino acid residue 254 is a substitution with threonine. In an embodiment, the amino acid substitution at amino acid residue 256 is a substitution with serine, arginine, glutamine, glutamic acid, aspartic acid, or threonine. In an embodiment, the amino acid substitution at amino acid residue 309 is a substitution with proline. In an embodiment, the amino acid substitution at amino acid residue 311 is a substitution with serine. In an embodiment, the amino acid substitution at amino acid residue 385 is a substitution with arginine, aspartic acid, serine, threonine, histidine, lysine, alanine or glycine. In an embodiment, the amino acid substitution at amino acid residue 386 is a substitution with threonine, proline, aspartic acid, serine, lysine, arginine, isoleucine, or methionine. In an embodiment, the amino acid substitution at amino acid residue 387 is a substitution with arginine, proline, histidine, serine, threonine, or alanine. In an embodiment, the amino acid substitution at amino acid residue 389 is a substitution with proline, serine or asparagine. In an embodiment, the amino acid substitution at amino acid residue 433 is a substitution with arginine, serine, isoleucine, proline, or glutamine. In an embodiment, the amino acid substitution at amino acid residue 434 is a substitution with histidine, phenylalanine, or tyrosine.

In some embodiments, the modified humanized antibodies of the invention comprise an IgG constant region that contains one or more mutations at amino acid residue 252, 254, 256, 433, 434, or 436. In an embodiment, the IgG constant region includes a triple M252Y/S254T/T256E mutation or YTE mutation. In another embodiment, the IgG constant region includes a triple H433K/N434F/Y436H mutation or KFH mutation. In a further embodiment, the IgG constant region includes an YTE and KFH mutation in combination.

In some embodiments, the modified humanized antibodies of the invention comprise an IgG constant region that contains one or more mutations at amino acid residues 250, 253, 307, 310, 380, 428, 433, 434, and 435. Illustrative mutations include T250Q, M428L, T307A, E380A, I253A, H310A, M428L, H433K, N434A, N434F, N434S, and H435A. In an embodiment, the IgG constant region comprises a M428L/N434S mutation or LS mutation. In another embodiment, the IgG constant region comprises a T250Q/M428L mutation or QL mutation. In another embodiment, the IgG constant region comprises an N434A mutation. In another embodiment, the IgG constant region comprises a T307A/E380A/N434A mutation or AAA mutation. In another embodiment, the IgG constant region comprises an I253A/H310A/H435A mutation or IHH mutation. In another embodiment, the IgG constant region comprises a H433K/N434F mutation. In another embodiment, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al Journal of Immunology. (2015), 194(11):5497-508, and U.S. Pat. No. 7,083,784, the entire contents of which are hereby incorporated by reference.

Amino acid modifications can be made by methods which are well known and routine for the skilled artisan. For example, but not by way of limitation, amino acid substitutions may be accomplished using any well-known PCR-based technique and site-directed mutagenesis (see, for example, Zoller and Smith, Nucl. Acids Res. 10:6487-6500, 1982; Kunkel, Proc. Natl. Acad. Sci USA 82:488, 1985, which are hereby incorporated by reference in their entireties). Additional exemplary mutations in the IgG constant region are described Borrok, et al. J Pharm Sci. 2017 April; 106(4):1008-1017, the entire contents of which are hereby incorporated by reference (e.g. L234F/L235E/P331S; L234F/L235Q/K322Q/M252Y/S254T/T256E).

In various embodiments, the one or more mutations in the IgG constant region increases affinity for the neonatal Fc receptor (FcRn). In some embodiments, the one or more mutations in the IgG constant region increases affinity for FcRn at a pH of about 6.0, about 6.1, about 6.2, about 6.3, about 6.4, about 6.5, about 6.6, about 6.7, about 6.8, about 6.9, or about 7.0. In various embodiments, the affinity for FcRn of the humanized antibodies comprising the modified IgG constant region is increased by about 10%, about 20%, about 30%, about 40%, about 50%, about 60%, about 70%, about 80%, about 90%, about 100%, about 2-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 150-fold or about 200-fold, or more, compared to humanized antibody comprising the wild-type IgG constant region. Affinity for FcRn can be measured by surface plasmon resonance (SPR) measurement using, for example, a BIAcore 2000 (BIAcore Inc.) as described in Popov et al., Mol. Immunol., 33:493-502, 1996 and Karlsson et al., J. Immunol. Methods, 145:229-240, 1991, both of which are incorporated by reference in their entireties. In this method, FcRn molecules are coupled to a BIAcore sensor chip (e.g., CM5 chip by Pharmacia) and the binding of modified IgG to the immobilized FcRn is measured at a certain flow rate to obtain sensorgrams using BIA evaluation 2.1 software, based on which on- and off-rates of the modified IgG constant regions, or fragments thereof, to FcRn can be calculated.

In various embodiments, the present modified humanized antibodies comprising the modified IgG constant region are stabilized and have extended in vivo half-lives. In various embodiments, the half-lives of the humanized antibodies comprising the modified IgG constant region is increased by about 10%, about 20%, about 30%, about 40%, about 50%, about 60%, about 70%, about 80%, about 90%, about 100%, about 2-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 150-fold or about 200-fold, or more, compared to humanized antibody comprising the wild-type IgG constant region. The half-life of an antibody comprising modified IgG or fragments thereof can be measured, for example, by pharmacokinetic studies according to the method described by Kim et al. (Eur. J. Immunol. 24:542, 1994), which is incorporated by reference herein in its entirety. According to this method, radiolabeled antibodies comprising modified IgG or fragments thereof is injected intravenously into mice and its plasma concentration is periodically measured as a function of time, for example, at 3 minutes to 72 hours after the injection. The clearance curve thus obtained should be biphasic, that is, α-phase and β-phase. For the determination of the in vivo half-life of the antibodies comprising modified IgGs or fragments thereof, the clearance rate in β-phase is calculated and compared with that of antibodies comprising the wild-type IgG.

In various embodiments, the modified humanized antibodies of the invention exhibit enhanced in vivo half-life thereby permitting lower effective dosages and/or less frequent dosing of the therapeutic antibodies relative to antibodies comprising wild-type IgG constant regions.

In various embodiments, the modified humanized antibodies of the invention exhibit enhanced bioavailability to various tissues or organs relative to antibodies comprising wild-type IgG constant regions. In various embodiments, the modified humanized antibodies of the invention exhibit enhanced bioavailability to, without limitation, the lungs, heart, pancreas, liver, kidney, bladder, stomach, large or small intestine, respiratory tract, lymph nodes, nervous tissue (central and/or peripheral nervous tissue), muscle, epidermis, bone, cartilage, joints, blood vessels, bone marrow, prostate, ovary, uterine, tumor or cancer tissue, etc.

In various embodiments, the modified humanized antibodies of the invention result in reduced side effects relative to antibodies comprising wild-type IgG constant regions.

### Humanization of Antibodies

In one aspect, the present invention is directed to a modified humanized 1B7 antibody and a humanized 11E6 antibody that bind a pertussis toxin protein. In various embodiments, a modified humanized antibody is a non-human antibody that has been altered to increase its similarity to a human antibody. In some embodiments, a modified humanized antibody is a genetically engineered antibody in which at least one CDR (or functional fragment thereof) from a non-human, e.g. mouse, antibody (“donor antibody”, which can also be rat, hamster or other non-human species) is grafted onto a human antibody (“acceptor antibody”). In some embodiments, more than one mouse CDR is grafted (e.g., all six mouse CDRs are grafted). The sequence of the acceptor antibody can be, for example, a mature human antibody sequence (or fragment thereof), a consensus sequence of a human antibody sequence (or fragment thereof), or a germline region sequence (or fragment thereof). Thus, in some embodiments, a modified humanized antibody may be an antibody having one or more CDRs from a donor antibody and variable region framework (FR). The FR may form part of a constant region within a human antibody.

In addition, in order to retain high binding affinity, amino acids in the human acceptor sequence may be replaced by the corresponding amino acids from the donor sequence, for example where: (1) the amino acid is in a CDR; (2) the amino acid is in the human framework region (e.g., the amino acid is immediately adjacent to one of the CDRs). See, U.S. Pat. Nos. 5,530,101 and 5,585,089, incorporated herein by reference, which provide detailed instructions for construction of humanized antibodies. Indeed, this selection of residues in, for example, the human framework region is often central to a humanized antibody's desirability. Although humanized antibodies often incorporate all six CDRs (e.g., as defined by Kabat, but often also including hypervariable loop H1 as defined by Chothia) from a mouse antibody, they can also be made with fewer mouse CDRs and/or less than the complete mouse CDR sequence (e.g. a functional fragment of a CDR).

In various embodiments, the humanized light chain variable region is fused to a light chain constant region (e.g. human kappa or a lambda light chain). In various embodiments, the humanized heavy chain variable region is fused to a heavy chain constant region, including various allotypes and isotypes of each. For example, the heavy chain constant region can be derived from any immunoglobulin type (e.g. IgG, IgM, IgA, IgD, or IgE). In some embodiments, IgG is used. For IgG, the constant region can come from IgG1, IgG2, IgG3, or IgG4. In some embodiments, IgG1 is used. In some embodiments, the IgG1 constant region comprises one or more mutations as described herein. Moreover, there are many isotypes of each IgG that can be chosen, some are naturally occurring and some are derivatives of naturally occurring isotypes. The type of IgG that is chosen will determine the effector functions of the antibody (e.g. opsonophagocytosis, complement fixation, etc.).

In one aspect, the present invention is directed to a modified humanized 1B7 antibody that binds a pertussis toxin protein, and comprises an immunoglobulin heavy chain variable region and an immunoglobulin light chain variable region.

In various embodiments, the immunoglobulin heavy chain variable region comprises:

a CDR_{H1} comprising an amino acid sequence selected from GYKFTSYWMH (SEQ ID NO:26);

a CDR_{H2} comprising an amino acid sequence selected from NIFPGSGSTNYDEKFNS (SEQ ID NO:27) and NIFPGSGSTNYAQKFQG (SEQ ID NO:28); and/or

a CDR_{H3} comprising an amino acid sequence selected from WLSGAYFDY (SEQ ID NO:29).

In various embodiments, the modified humanized 1B7 antibody comprises 1, or 2, or 3 of CDR_{H1}, CDR_{H2}, and CDR_{H3} as described above.

In an embodiment, the modified humanized 1B7 antibody comprises an immunoglobulin heavy chain variable region comprising a CDR_{H1} comprising the amino acid sequence of SEQ ID NO:26, a CDR_{H2} comprising the amino acid sequence of SEQ ID NO:27, and a CDR_{H3} comprising the amino acid sequence of SEQ ID NO: 29.

In an embodiment, the modified humanized 1B7 antibody comprises an immunoglobulin heavy chain variable region comprising a CDR_{H1} comprising the amino acid sequence of SEQ ID NO:26, a CDR_{H2} comprising the amino acid sequence of SEQ ID NO:28, and a CDR_{H3} comprising the amino acid sequence of SEQ ID NO: 29.

In various embodiments, the immunoglobulin light chain variable region comprises:

a CDR_{L1} comprising an amino acid sequence selected from SASSSVSFMY (SEQ ID NO:30), RASSSVSFMY (SEQ ID NO:31), and RASSIVSFLY (SEQ ID NO:32);

a CDR_{L2} comprising an amino acid sequence selected from LTSNLPS (SEQ ID NO:33) and LASNLPS (SEQ ID NO:34); and/or

a CDR_{L3} comprising an amino acid sequence selected from QQWSSHPPT (SEQ ID NO:35).

In various embodiments, the modified humanized 1B7 antibody comprises 1, or 2, or 3 of CDR_{L1}, CDR_{L2}, and CDR_{L3} as described above.

In an embodiment, the modified humanized 1B7 antibody comprises an immunoglobulin light chain variable region comprising a CDR_{L1} comprising the amino acid sequence of SEQ ID NO:30, a CDR_{L2} comprising the amino acid sequence of SEQ ID NO:33, and a CDR_{L3} comprising the amino acid sequence of SEQ ID NO: 35.

In an embodiment, the modified humanized 1B7 antibody comprises an immunoglobulin light chain variable region comprising a CDR_{L1} comprising the amino acid sequence of SEQ ID NO:31, a CDR_{L2} comprising the amino acid sequence of SEQ ID NO:33, and a CDR_{L3} comprising the amino acid sequence of SEQ ID NO: 35.

In an embodiment, the modified humanized 1B7 antibody comprises an immunoglobulin light chain variable region comprising a CDR_{L1} comprising the amino acid sequence of SEQ ID NO:32, a CDR_{L2} comprising the amino acid sequence of SEQ ID NO:34, and a CDR_{L3} comprising the amino acid sequence of SEQ ID NO: 35.

In some embodiments, the immunoglobulin heavy chain variable region comprises an amino acid sequence selected from:

1B7:
(SEQ ID NO: 1)
QVQLQQPGSELVRPGASVKLSCKASGYKFTSYWMHWVKQRPGQGLEWIG
NIFPGSGSTNYDEKFNSKATLTVDTSSNTAYMQLSSLTSEDSAVYYCTR
WLSGAYFDYWGQGTTLTVSS,
cdr1B7:
(SEQ ID NO: 2)
QVQLVQSGAEVKKPGASVKVSCKASGYKFTSYWMHWVRQAPGQGLEWIG
NIFPGSGSTNYDEKFNSRVTLTVDTSTSTAYMELSSLRSEDTAVYYCTR
WLSGAYFDYWGQGTTVTVSS,
abb1B7:
(SEQ ID NO: 3)
QVQLVQSGAEVKKPGASVKVSCKASGYKFTSYWMHWVRQAPGQGLEWIG
NIFPGSGSTNYAQKFQGRVTLTVDTSTSTAYMELSSLRSEDTAVYYCTR
WLSGAYFDYWGQGTTVTVSS,
sdr1B7:
(SEQ ID NO: 4)
QVQLVQSGAEVKKPGASVKVSCKASGYKFTSYWMHWVRQAPGQGLEWIG
NIFPGSGSTNYAQKFQGRVTLTVDTSTSTAYMELSSLRSEDTAVYYCTR
WLSGAYFDYWGQGTTVTVSS,
fra1B7:
(SEQ ID NO: 5)
QVQLQQSGSELKKPGASVKISCKASGYKFTSYWMHWVKQRPGQGLEWIG
NIFPGSGSTNYDEKFNSRVTLTVDTSTSTAYMELSSLRSEDTAVYYCTR
WLSGAYFDYWGQGTTLTVSS,
and
ven1B7:
(SEQ ID NO: 6)
QVQLVQSGAELVKPGASVKLSCKASGYKFTSYWMHWVKQRPGQGLEWIG
NIFPGSGSTNYDEKFNSKATLTVDTSTSTAYMELSSLRSEDTAVYYCTR
WLSGAYFDYWGQGTTLTVSS;

and
the immunoglobulin light chain variable region comprises an amino acid sequence selected from:

1B7:
(SEQ ID NO: 7)
QIVLTQSPALMSASPGEKVTMTCSASSSVSFMYWYQQKPRSSPKPWIY
LTSNLPSGVPARFSGSGSGTSYSLTISSMEAEDAATYYCQQWSSHPPT
FGSGTKLEIK,
cdr1B7:
(SEQ ID NO: 8)
QIVLTQSPDFQSVTPKEKVTITCSASSSVSFMYWYQQKPDQSPKPLIY
LTSNLPSGVPARFSGSGSGTSYTLTINSLEAEDAATYYCQQWSSHPPT
FGSGTKVEIK,
abb1B7:
(SEQ ID NO: 9)
QIVLTQSPDFQSVTPKEKVTITCRASSSVSFMYWYQQKPDQSPKPLIY
LTSNLPSGVPARFSGSGSGTDYTLTINSLEAEDAATYYCQQWSSHPPT
FGSGTKVEIK,
sdr1B7:
(SEQ ID NO: 10)
QIVLTQSPDFQSVTPKEKVTITCRASSIVSFLYWYQQKPDQSPKPLIY
LASNLPSGVPARFSGSGSGTDYTLTINSLEAEDAATYYCQQWSSHPPT
FGSGTKVEIK,
fra1B7:
(SEQ ID NO: 11)
QIVLTQSPATLSVSPGERVTLTCSASSSVSFMYWYQQKPGRAPKPLIY
LTSNLPSGVPARFSGSGSGTSYTLTINSLEAEDAATYYCQQWSSHPPT
FGSGTKLEIK,
and
ven1B7:
(SEQ ID NO: 12)
QIVLTQSPDFMSATPGEKVTMTCSASSSVSFMYWYQQKPRQSPKPWIY
LTSNLPSGVPARFSGSGSGTDYTLTINSMEAEDAATYYCQQWSSHPPT
FGSGTKLEIK.

Any one of the disclosed 1B7 heavy chains can be paired with any of the disclosed 1B7 light chains. By way of illustration, the following pairs can be incorporated into an antibody of the present compositions and methods: SEQ ID NO: 1/SEQ ID NO: 7; SEQ ID NO: 1/SEQ ID NO: 8; SEQ ID NO: 1/SEQ ID NO: 9; SEQ ID NO: 1/SEQ ID NO: 10; SEQ ID NO: 1/SEQ ID NO: 11; SEQ ID NO: 1/SEQ ID NO: 12; SEQ ID NO: 2/SEQ ID NO: 7; SEQ ID NO: 2/SEQ ID NO: 8; SEQ ID NO: 2/SEQ ID NO: 9; SEQ ID NO: 2/SEQ ID NO: 10; SEQ ID NO: 2/SEQ ID NO: 11; SEQ ID NO: 2/SEQ ID NO: 12; SEQ ID NO: 3/SEQ ID NO: 7; SEQ ID NO: 3/SEQ ID NO: 8; SEQ ID NO: 3/SEQ ID NO: 9; SEQ ID NO: 3/SEQ ID NO: 10; SEQ ID NO: 3/SEQ ID NO: 11; SEQ ID NO: 3/SEQ ID NO: 12; SEQ ID NO: 4/SEQ ID NO: 7; SEQ ID NO: 4/SEQ ID NO: 8; SEQ ID NO: 4/SEQ ID NO: 9; SEQ ID NO: 4/SEQ ID NO: 10; SEQ ID NO: 4/SEQ ID NO: 11; SEQ ID NO: 4/SEQ ID NO: 12; SEQ ID NO: 5/SEQ ID NO: 7; SEQ ID NO: 5/SEQ ID NO: 8; SEQ ID NO: 5/SEQ ID NO: 9; SEQ ID NO: 5/SEQ ID NO: 10; SEQ ID NO: 5/SEQ ID NO: 11; SEQ ID NO: 5/SEQ ID NO: 12; SEQ ID NO: 6/SEQ ID NO: 7; SEQ ID NO: 6/SEQ ID NO: 8; SEQ ID NO: 6/SEQ ID NO: 9; SEQ ID NO: 6/SEQ ID NO: 10; SEQ ID NO: 6/SEQ ID NO: 11; and SEQ ID NO: 6/SEQ ID NO: 12.

In one embodiment, the modified humanized 1B7 antibody comprises an immunoglobulin heavy chain variable region comprising the amino acid sequence of SEQ ID NO: 2, and an immunoglobulin light chain variable region comprising the amino acid sequence of SEQ ID NO:8.

In one embodiment, the modified humanized 1B7 antibody comprises an immunoglobulin heavy chain variable region comprising the amino acid sequence of SEQ ID NO: 3, and an immunoglobulin light chain variable region comprising the amino acid sequence of SEQ ID NO:9.

In one embodiment, the modified humanized 1B7 antibody comprises an immunoglobulin heavy chain variable region comprising the amino acid sequence of SEQ ID NO: 4, and an immunoglobulin light chain variable region comprising the amino acid sequence of SEQ ID NO:10.

In one embodiment, the modified humanized 1B7 antibody comprises an immunoglobulin heavy chain variable region comprising the amino acid sequence of SEQ ID NO: 5, and an immunoglobulin light chain variable region comprising the amino acid sequence of SEQ ID NO:11.

In one embodiment, the modified humanized 1B7 antibody comprises an immunoglobulin heavy chain variable region comprising the amino acid sequence of SEQ ID NO: 6, and an immunoglobulin light chain variable region comprising the amino acid sequence of SEQ ID NO:12.

In other embodiments, the modified humanized 1B7 antibody comprises an immunoglobulin heavy chain variable region comprising an amino acid sequence having at least about 50% identity, about 51% identity, about 52% identity, about 53% identity, about 54% identity, about 55% identity, about 56% identity, about 57% identity, about 58% identity, about 59% identity, about 60% identity, about 61% identity, about 62% identity, about 63% identity, about 64% identity, about 65% identity, about 66% identity, about 67% identity, about 68% identity, about 69% identity, about 70% identity, about 71% identity, about 72% identity, about 73% identity, about 74% identity, about 75% identity, about 76% identity, about 77% identity, about 78% identity, about 79% identity, about 80% identity, about 81% identity, about 82% identity, about 83% identity, about 84% identity, about 85% identity, about 86% identity, about 87% identity, about 88% identity, about 89% identity, about 90% identity, about 93% identity, about 95% identity, about 97% identity, about 98% identity, or about 99% identity to the entire variable region, the complementarity determining regions, or the framework region sequence of SEQ ID NO:1, SEQ ID NO:2, SEQ ID NO:3, SEQ ID NO:4, SEQ ID NO:5, or SEQ ID NO:6.

In other embodiments, the modified humanized 1B7 antibody comprises an immunoglobulin light chain variable region comprising an amino acid sequence having at least about 50% identity, about 51% identity, about 52% identity, about 53% identity, about 54% identity, about 55% identity, about 56% identity, about 57% identity, about 58% identity, about 59% identity, about 60% identity, about 61% identity, about 62% identity, about 63% identity, about 64% identity, about 65% identity, about 66% identity, about 67% identity, about 68% identity, about 69% identity, about 70% identity, about 71% identity, about 72% identity, about 73% identity, about 74% identity, about 75% identity, about 76% identity, about 77% identity, about 78% identity, about 79% identity, about 80% identity, about 81% identity, about 82% identity, about 83% identity, about 84% identity, about 85% identity, about 86% identity, about 87% identity, about 88% identity, about 89% identity, or about 90% identity to the entire variable region, the complementarity determining regions, or the framework region sequence of SEQ ID NO:7, SEQ ID NO:8, SEQ ID NO:9, SEQ ID NO:10, SEQ ID NO:11, or SEQ ID NO:12.

In one aspect, the present invention is directed to a modified humanized 11E6 antibody that binds a pertussis toxin protein, and comprises an immunoglobulin heavy chain variable region and an immunoglobulin light chain variable region.

In various embodiments, the immunoglobulin heavy chain variable region comprises:

a CDR_{H1} comprising an amino acid sequence selected from GFTFTDYYVS (SEQ ID NO:36),

a CDR_{H2} comprising an amino acid sequence selected from FIRNKVNGYTTEFSSSVKG (SEQ ID NO:37) and FIRNKVNGYTTEFAASVRG (SEQ ID NO:38), and/or

a CDR_{H3} comprising an amino acid sequence selected from VSYYGRGWYFDY (SEQ ID NO:39).

In various embodiments, the modified humanized 11E6 antibody comprises 1, or 2, or 3 of CDR_{H1}, CDR_{H2}, and CDR_{H3} as described above.

In an embodiment, the modified humanized 11E6 antibody comprises an immunoglobulin heavy chain variable region comprising a CDR_{H1} comprising the amino acid sequence of SEQ ID NO:36, a CDR_{H2} comprising the amino acid sequence of SEQ ID NO:37, and a CDR_{H3} comprising the amino acid sequence of SEQ ID NO: 39.

In an embodiment, the modified humanized 11E6 antibody comprises an immunoglobulin heavy chain variable region comprising a CDR_{H1} comprising the amino acid sequence of SEQ ID NO:36, a CDR_{H2} comprising the amino acid sequence of SEQ ID NO:38, and a CDR_{H3} comprising the amino acid sequence of SEQ ID NO: 39.

In various embodiments, the immunoglobulin light chain variable region comprises:

a CDR_{L1} comprising an amino acid sequence selected from RASQDIDNYLS (SEQ ID NO:40),

a CDR_{L2} comprising an amino acid sequence selected from YTSRLHS (SEQ ID NO:41), and/or

a CDR_{L3} comprising an amino acid sequence selected from QQGNTFPWT (SEQ ID NO:42).

In various embodiments, the modified humanized 11E6 antibody comprises 1, or 2, or 3 of CDR_{L1}, CDR_{L2}, and CDR_{L3} as described above.

In an embodiment, the modified humanized 11E6 antibody comprises an immunoglobulin light chain variable region comprising a CDR_{L1} comprising the amino acid sequence of SEQ ID NO:40, a CDR_{L2} comprising the amino acid sequence of SEQ ID NO:41, and a CDR_{L3} comprising the amino acid sequence of SEQ ID NO: 42.

In some embodiments, the immunoglobulin heavy chain variable region comprises an amino acid sequence selected from:

11E6:
(SEQ ID NO: 13)
EVKVVESGGGLVQPGGSLRLSCTTSGFTFTDYYVSWVRQPPGKALEWLGFIRNKVNG
YTT
EFSSSVKGRFTISRDNSQSILYLQMNTLRVEDSATYYCARVSYYGRGWYFDYWGQGT
TLT
VSS
cdr11E6:
(SEQ ID NO: 14)
EVQVVESGGGLVQPGRSLRLSCTTSGFTFTDYYVSWVRQAPGKALEWLGFIRNKVNG
YTT
EFSSSVKGRFTISRDNSKSILYLQMNSLKIEDTAVYYCARVSYYGRGWYFDYWGQGTT
VT
VSS
abb11E6:
(SEQ ID NO: 15)
EVQVVESGGGLVQPGRSLRLSCTTSGFTFTDYYVSWVRQAPGKALEWVGFIRNKVNG
YTT
EFAASVRGRFTISRDNSKSILYLQMNSLKIEDTAVYYCARVSYYGRGWYFDYWGQGT
TVT
VSS
sdr11E6:
(SEQ ID NO: 16)
EVQVVESGGGLVQPGRSLRLSCTTSGFTFTDYYVSWVRQAPGKALEWVGFIRNKVNG
YTT
EFAASVRGRFTISRDNSKSILYLQMNSLKIEDTAVYYCARVSYYGRGWYFDYWGQGT
TVT
VSS
fra11E6:
(SEQ ID NO: 17)
EVQVVESGGGLVQPGGSLRLSCTTSGFTFTDYYVSWVRQPPGKALEWLGFIRNKVNG
YTT
EFSSSVKGRFTISRDNSKSTLYLQMNTLRVDDTAVYYCARVSYYGRGWYFDYWGQG
TTLT
VSS
and
ven11E6:
(SEQ ID NO: 18)
EVQVVESGGGLVQPGRSLRLSCTTSGFTFTDYYVSWVRQAPGKALEWLGFIRNKVNG
YTT
EFSSSVKGRFTISRDNSKSILYLQMNSLKIEDTAVYYCARVSYYGRGWYFDYWGQGTT
LT
VSS;

and
the immunoglobulin light chain variable region comprises an amino acid sequence selected from:

11E6:
(SEQ ID NO: 19)
DIVMTQSTSSLSASLGDRVTISCRASQDIDNYLSWFQQKPDGTVKLLIY
YTSRLHSGVPSRFSGSGSGTDYSLTISSLDQEDIATYFCQQGNTFPWTF
GGGTKLEIK
cdr11E6:
(SEQ ID NO: 20)
DIVMTQSPSSLSASVGDRVTISCRASQDIDNYLSWFQQKPGGTVKLLIY
YTSRLHSGVPSRFSGSGSGTDYTLTISSLQPEDIATYFCQQGNTFPWTF
GGGTKVEIK
abb11E6:
(SEQ ID NO: 21)
DIVMTQSPSSLSASVGDRVTITCRASQDIDNYLSWFQQKPGGTVKLLIY
YTSRLHSGVPSRFSGSGSGTDYTLTISSLQPEDIATYFCQQGNTFPWTF
GGGTKVEIK
sdr11E6:
(SEQ ID NO: 22)
DIVMTQSPSSLSASVGDRVTITCRASQDIDNYLSWFQQKPGGTVKLLIY
YTSRLHSGVPSRFSGSGSGTDYTLTISSLQPEDIATYFCQQGNTFPWTF
GGGTKVEIK
fra11E6:
(SEQ ID NO: 23)
DIVMTQSPSSLSASVGDRVTISCRASQDIDNYLSWFQQKPGGTVKLLIY
YTSRLHSGVPSRFSGSGSGTDYTLTISSLQPEDIATYFCQQGNTFPWTF
GGGTKLEIK
ven11E6:
(SEQ ID NO: 24)
DIVMTQSPSSLSASVGDRVTISCRASQDIDNYLSWFQQKPGGTVKLLIY
YTSRLHSGVPSRFSGSGSGTDYTLTISSLQPEDIATYFCQQGNTFPWTF
GGGTKLEIK.

Any one of the disclosed 11E6 heavy chains can be paired with any of the disclosed 11E6 light chains. By way of illustration, the following pairs can be incorporated into an antibody of the present compositions and methods: SEQ ID NO: 13/SEQ ID NO: 19; SEQ ID NO: 13/SEQ ID NO: 20; SEQ ID NO: 13/SEQ ID NO: 21; SEQ ID NO: 13/SEQ ID NO: 22; SEQ ID NO: 13/SEQ ID NO: 23; SEQ ID NO: 13/SEQ ID NO: 24; SEQ ID NO: 14/SEQ ID NO: 19; SEQ ID NO: 14/SEQ ID NO: 20; SEQ ID NO: 14/SEQ ID NO: 21; SEQ ID NO: 14/SEQ ID NO: 22; SEQ ID NO: 14/SEQ ID NO: 23; SEQ ID NO: 14/SEQ ID NO: 24; SEQ ID NO: 15/SEQ ID NO: 19; SEQ ID NO: 15/SEQ ID NO: 20; SEQ ID NO: 15/SEQ ID NO: 21; SEQ ID NO: 15/SEQ ID NO: 22; SEQ ID NO: 15/SEQ ID NO: 23; SEQ ID NO: 15/SEQ ID NO: 24; SEQ ID NO: 16/SEQ ID NO: 19; SEQ ID NO: 16/SEQ ID NO: 20; SEQ ID NO: 16/SEQ ID NO: 21; SEQ ID NO: 16/SEQ ID NO: 22; SEQ ID NO: 16/SEQ ID NO: 23; SEQ ID NO: 16/SEQ ID NO: 24; SEQ ID NO: 17/SEQ ID NO: 19; SEQ ID NO: 17/SEQ ID NO: 20; SEQ ID NO: 17/SEQ ID NO: 21; SEQ ID NO: 17/SEQ ID NO: 22; SEQ ID NO: 17/SEQ ID NO: 23; SEQ ID NO: 17/SEQ ID NO: 24; SEQ ID NO: 18/SEQ ID NO: 19; SEQ ID NO: 18/SEQ ID NO: 20; SEQ ID NO: 18/SEQ ID NO: 21; SEQ ID NO: 18/SEQ ID NO: 22; SEQ ID NO: 18/SEQ ID NO: 23; and SEQ ID NO: 18/SEQ ID NO: 24.

In one embodiment, the modified humanized 11E6 antibody comprises an immunoglobulin heavy chain variable region comprising the amino acid sequence of SEQ ID NO: 14, and an immunoglobulin light chain variable region comprising the amino acid sequence of SEQ ID NO:20.

In one embodiment, the modified humanized 11E6 antibody comprises an immunoglobulin heavy chain variable region comprising the amino acid sequence of SEQ ID NO: 15, and an immunoglobulin light chain variable region comprising the amino acid sequence of SEQ ID NO:21.

In one embodiment, the modified humanized 11E6 antibody comprises an immunoglobulin heavy chain variable region comprising the amino acid sequence of SEQ ID NO: 16, and an immunoglobulin light chain variable region comprising the amino acid sequence of SEQ ID NO:22.

In one embodiment, the modified humanized 11E6 antibody comprises an immunoglobulin heavy chain variable region comprising the amino acid sequence of SEQ ID NO:17, and an immunoglobulin light chain variable region comprising the amino acid sequence of SEQ ID NO:23.

In one embodiment, the modified humanized 11E6 antibody comprises an immunoglobulin heavy chain variable region comprising the amino acid sequence of SEQ ID NO:18, and an immunoglobulin light chain variable region comprising the amino acid sequence of SEQ ID NO:24.

In other embodiments, the modified humanized 11E6 antibody comprises an immunoglobulin heavy chain variable region comprising an amino acid sequence having at least about 50% identity, about 51% identity, about 52% identity, about 53% identity, about 54% identity, about 55% identity, about 56% identity, about 57% identity, about 58% identity, about 59% identity, about 60% identity, about 61% identity, about 62% identity, about 63% identity, about 64% identity, about 65% identity, about 66% identity, about 67% identity, about 68% identity, about 69% identity, about 70% identity, about 71% identity, about 72% identity, about 73% identity, about 74% identity, about 75% identity, about 76% identity, about 77% identity, about 78% identity, about 79% identity, about 80% identity, about 81% identity, about 82% identity, about 83% identity, about 84% identity, about 85% identity, about 86% identity, about 87% identity, about 88% identity, about 89% identity, or about 90% identity to the entire variable region, the complementarity determining regions, or the framework region sequence of SEQ ID NO:13, SEQ ID NO:14, SEQ ID NO:15, SEQ ID NO:16, SEQ ID NO:17, and SEQ ID NO:18.

In other embodiments, the modified humanized 11E6 antibody comprises an immunoglobulin light chain variable region comprising an amino acid sequence having at least about 50% identity, about 51% identity, about 52% identity, about 53% identity, about 54% identity, about 55% identity, about 56% identity, about 57% identity, about 58% identity, about 59% identity, about 60% identity, about 61% identity, about 62% identity, about 63% identity, about 64% identity, about 65% identity, about 66% identity, about 67% identity, about 68% identity, about 69% identity, about 70% identity, about 71% identity, about 72% identity, about 73% identity, about 74% identity, about 75% identity, about 76% identity, about 77% identity, about 78% identity, about 79% identity, about 80% identity, about 81% identity, about 82% identity, about 83% identity, about 84% identity, about 85% identity, about 86% identity, about 87% identity, about 88% identity, about 89% identity, or about 90% identity to the entire variable region, the complementarity determining regions, or the framework region sequence of SEQ ID NO:19, SEQ ID NO:20, SEQ ID NO:21, SEQ ID NO:22, SEQ ID NO:23, and SEQ ID NO:24.

Homology or identity may be determined in various ways that are within the skill in the art, for instance, using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87, 2264-2268; Altschul, (1993) J. MOL. EVOL. 36, 290-300; Altschul et al., (1997) NUCLEIC ACIDS RES. 25, 3389-3402, incorporated by reference) are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases see Altschul et al., (1994) NATURE GENETICS 6, 119-129 which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89, 10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/ 11 for proteins; −E, Cost to extend gap [Integer]: default=2 for nucleotides/ 1 for proteins; −q, Penalty for nucleotide mismatch [Integer]: default=−3; −r, reward for nucleotide match [Integer]: default=1; −e, expect value [Real]: default=10; —W, wordsize [Integer]: default=11 for nucleotides/ 28 for megablast/ 3 for proteins; −y, Dropoff (X) for blast extensions in bits: default=20 for blastn/7 for others; —X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In each of the foregoing embodiments, it is contemplated herein that the immunoglobulin heavy chain variable region sequences and/or light chain variable region sequences may contain amino acid alterations (e.g., amino acid substitutions, deletions, or insertions) relative to SEQ ID NOs:1-24. For example, the immunoglobulin heavy chain variable region sequences and/or light chain variable region sequences may contain from about 1 to about 50 mutations, from about 1 to about 40 mutations, from about 1 to about 35 mutations, from about 1 to about 30 mutations, about 1 to about 25 mutations, from about 1 to about 20 mutations, about 1 to about 15 mutations, or from about 1 to about 10 mutations independently selected from substitutions, deletions, or insertions with respect to SEQ ID NOs:1-24. In various embodiments, the immunoglobulin heavy chain variable region sequences and/or light chain variable region sequences have about 1 mutation, about 2 mutations, about 3 mutations, about 4 mutations, about 5 mutations, about 6 mutations, about 7 mutations, about 8 mutations, about 9 mutations, about 10 mutations, about 11 mutations, about 12 mutations, about 13 mutations, about 14 mutations, about 15 mutations, about 16 mutations, about 17 mutations, about 18 mutations, about 19 mutations, about 20 mutations, about 21 mutations, about 22 mutations, about 23 mutations, about 24 mutations, about 25 mutations, about 26 mutations, about 27 mutations, about 28 mutations, about 29 mutations, about 30 mutations, about 31 mutations, about 32 mutations, about 33 mutations, about 34 mutations, about 35 mutations, about 36 mutations, about 37 mutations, about 38 mutations, about 39 mutations, about 40 mutations, about 41 mutations, about 42 mutations, about 43 mutations, about 44 mutations, about 45 mutations, about 46 mutations, about 47 mutations, about 48 mutations, about 49 mutations, or about 50 mutations, relative to SEQ ID NOs:1-24. Illustrative amino acids that may be incorporated include a hydrophilic amino acid residue, which may include a polar and positively charged hydrophilic residue selected from arginine (R) and lysine (K), a polar and neutral of charge hydrophilic residue selected from asparagine (N), glutamine (Q), serine (S), threonine (T), proline (P), and cysteine (C), a polar and negatively charged hydrophilic residue selected from aspartate (D) and glutamate (E), or an aromatic, polar and positively charged hydrophilic including histidine (H); a hydrophobic amino acid residue which may include a hydrophobic, aliphatic amino acid selected from glycine (G), alanine (A), leucine (L), isoleucine (I), methionine (M), or valine (V) or a hydrophobic, aromatic amino acid selected from phenylalanine (F), tryptophan (W), or tyrosine (Y).

The ability of an antibody to bind a specific epitope can be described by the equilibrium dissociation constant (K_{D}). In certain embodiments, the present invention provides a modified humanized 1B7 antibody that binds the pertussis toxin protein with a K_{D} of about 20 nM or lower, or about 15 nM or lower, or about 10 nM or lower, or about 5 nM or lower. In an embodiment, the modified humanized 1B7 antibody binds the pertussis toxin protein with a K_{D} of about 5 nM or lower or about 3 nM or lower. In illustrative embodiments, the modified humanized 1B7 antibody binds the pertussis toxin protein with a K_{D} of about 5 nM, about 4.5 nM, about 4 nM, about 3.5 nM, about 3 nM, about 2.5 nM, about 2 nM, about 1.5 nM, about 1 nM, or about 0.5 nM.

In certain embodiments, the present invention provides a modified humanized 11E6 antibody that binds the pertussis toxin protein with a K_{D} of about 20, about 19, or about 18, or about 17, or about 16, or about 15 nM or lower. In an embodiment, the modified humanized 11E6 antibody binds the pertussis toxin protein with a K_{D} of 12 nM or lower. In illustrative embodiments, the modified humanized 1B7 antibody binds the pertussis toxin protein with a K_{D} of about 15 nM, about 14.5 nM, about 14 nM, about 13.5 nM, about 13 nM, about 12.5 nM, about 12 nM, about 11.5 nM, about 11 nM, about 10.5 nM, about 10 nM, about 9.5 nM, about 9 nM, about 8.5 nM, about 8 nM, about 7.5 nM, about 7 nM, about 6.5 nM, about 6 nM, about 5.5 nM, about 5 nM, about 4.5 nM, about 4 nM, about 3.5 nM, about 3 nM, about 2.5 nM, about 2 nM, about 1.5 nM, about 1 nM, or about 0.5 nM.

In some embodiments, the modified humanized antibodies described herein compete with an antibody that is capable of binding a pertussis toxin protein. Where the modified humanized antibody competes with an antibody (competitor antibody) for binding a pertussis toxin protein, the modified humanized antibodies of the invention inhibit (completely or partially) binding of the competitor antibody to a measurable extent. The inhibition of binding may be measured by any of the methods known in the art. In general, a humanized antibody is considered to competitively inhibit binding of a competitor antibody (e.g., mouse 1B7 or 11E6 antibody as described by Sato et al., (1990), Infection and Immunity, 58(10): 3369-3374 or humanized 1B7 antibody as described by Maynard et al., U.S. Pat. No. 8,653,243, which are herein incorporated by reference in their entireties), if binding of the competitor antibody to the antigen is reduced by at least about 10%, at least about 20%, at least about 30%, at least about 40%, at least about 50%, at least about 60%, at least about 70%, at least about 80%, or at least about 90%, in the presence of the humanized antibody. Thus, in some embodiments, the antibody provided herein binds to a pertussis toxin protein competitively with a mouse 1B7 or 11E6 antibody as described by Sato et al., (1990), Infection and Immunity, 58(10): 3369-3374. In other embodiments, the antibody provided herein inhibits (completely or partially) the binding of a mouse 1B7 or 11E6 antibody. In some further embodiments, the antibody provided herein decreases the binding of a mouse 1B7 or 11E6 antibody in a competition assay by about 10%, about 20%, about 30%, about 40%, about 50%, about 60%, about 70%, about 80%, about 90% or about 100%.

Although the embodiments illustrated in the Examples may comprise pairs of variable regions, pairs of full length antibody chains, or pairs of CDR1, CDR2 and CDR3 regions, one from a heavy chain and one from a light chain, a skilled artisan will recognize that alternative embodiments may comprise single heavy chain variable regions or single light chain variable regions, single full length antibody chains, or CDR1, CDR2 and CDR3 regions from one antibody chain, either heavy or light.

### Production of Antibodies

Methods for producing antibodies of the invention are described herein. For example, DNA molecules encoding light chain variable and/or constant regions and/or heavy chain variable and/or constant regions can be chemically synthesized using the sequence information provided herein. Synthetic DNA molecules can be ligated to other appropriate nucleotide sequences, including, e.g., expression control sequences, to produce gene expression constructs encoding the desired antibodies. Alternatively, the sequences provided herein can be cloned out of hybridomas by hybridization techniques or polymerase chain reaction (PCR) techniques using synthetic nucleic acid probes.

Nucleic acids encoding desired antibodies can be incorporated (ligated) into expression vectors, which can be introduced into host cells through transfection, transformation, or transduction techniques. For example, nucleic acids encoding desired antibodies can be introduced into host cells by retroviral transduction. Illustrative host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, IgG enhancers, and various introns. The gene construct can be introduced into eukaryotic host cells using transfection, transformation, or transduction techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function. In some embodiments, a host cell is transfected with a single vector expressing a polypeptide expressing an entire, or part of, a heavy chain (e.g., a heavy chain variable and/or constant region) or a light chain (e.g., a light chain variable and/or constant region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

A polypeptide comprising an immunoglobulin heavy chain variable region (and/or constant region) or light chain variable region (and/or constant region) can be produced by growing a host cell transfected with an expression vector encoding such regions, under conditions that permit expression of the polypeptide. Following expression, the polypeptide can be harvested and purified using techniques well known in the art, e.g., affinity tags such as glutathione-S-transferase (GST) and histidine tags or by chromatography (by way of non-limiting example, based on size, charge, and/or specific binding).

A monoclonal antibody that binds the pertussis toxin protein, or an antigen-binding fragment of the antibody, can be produced by growing a host cell transfected, transformed or transduced with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment) can be harvested and purified using techniques well known in the art, e.g., Protein A, Protein G, affinity tags such as glutathione-S-transferase (GST) and histidine tags or by chromatography. It is within ordinary skill in the art to express the heavy chain and the light chain from a single expression vector or from two separate expression vectors.

### Antibody Modifications

There are standard methods for reducing or eliminating the antigenicity of antibodies and antibody fragments that are known in the art. When the antibodies are to be administered to a human, the antibodies preferably are “humanized” to reduce or eliminate antigenicity in humans. It is contemplated that the humanized antibodies have at least the same or substantially the same affinity for the antigen as the non-humanized mouse antibody from which it was derived.

However, it is noted that while humanization approaches are known in the art, some humanization approaches are hindered by reductions in affinity (e.g. relative to the original murine antibody). This may be, without wishing to be bound by theory, due to the fact that the CDRs are not maintained workable configuration by the human frameworks. In this case, a small number of changes to the human framework sequences are made. These individual amino acid changes improve the affinity without making significant deviations from the human antibody structure so that the antibodies continue to resemble human antibodies. In that way, the antibodies can be used as for therapeutic purposes in humans without inducing an immune response. The choice of amino acids to change and the specific changes to be made are part of the present invention.

In one humanization approach, chimeric proteins are created in which mouse immunoglobulin constant regions are replaced with human immunoglobulin constant regions. See, e.g., Morrison et al., 1984, PROC. NAT. ACAD. Su 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly). For example, in some embodiments, any one of SEQ ID NO: 1, or SEQ ID NO: 7, or SEQ ID NO: 13, or SEQ ID NO: 19 can be the variable regions that are paired with a human constant region.

In an approach known as CDR grafting, the CDRs of the light and heavy chain variable regions are grafted into frameworks from another species. For example, murine CDRs and non-CDR residues involved in antigen binding can be grafted into human sequences. Residues involved in maintaining the combining site structure and residues involved in maintaining V_{L}:V_{H} contact may also be grafted. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called grafting of abbreviated CDRs, abbreviated CDRs, as defined by Padlan et al., (1995) FASEB J 9:133-139, and non-CDR residues involved in antigen binding, are transplanted into a human sequence. Residues involved in maintaining the combining site structure and residues involved in maintaining V_{L}:V_{H} contact may also be grafted.

Other methods to reduce immunogenicity include “SDR-transfer,” “veneering,” and “Frankensteining.” See, e.g., Padlan et al., (1995) FASEB J 9:133-139, Wu et al., (1992) MOL IMMUNOL 29:1141-1146, and Padlan et al., (1991) MOL IMMUNOL 28:489-498. In the SDR-transfer approach, residues involved in antigen binding (i.e., the specificity-determining residues or SDRs) are transplanted into a human sequence. Residues involved in maintaining the combining site structure and residues involved in maintaining V_{L}:V_{H} contact may also be transplanted. In the veneering approach, the surface accessible amino acid residues in the murine antibody are replaced by amino acid residues more frequently found at the same positions in a human antibody. For example, the framework residues, which are exposed to solvent, are replaced with their homologues from a human sequence. The CDRs and non-CDR residues involved in antigen binding are preserved. In the Frankensteining approach, the CDRs are transplanted into a composite sequence constructed from the most similar human framework regions. Residues involved in maintaining the combining site structure and residues involved in maintaining V_{L}:V_{H} contact may also be transplanted.

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., N_{ATURE} 368:856-859, 1994; Fishwild et al., N_{ATURE} B_{IOTECHNOLOGY} 14:845-851, 1996; and Mendez et al., N_{ATURE} G_{ENETICS} 15:146-156, 1997. Human mAbs can also be prepared and optimized from phage display libraries by techniques referenced in, e.g., Knappik et al., J. M_{OL}. B_{IOL}. 296:57-86, 2000; and Krebs et al., J. Immunol. Meth. 254:67-84 2001).

If the antibody is for use as a therapeutic, it can be conjugated to an effector agent such as a small molecule or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

### Methods of Using Antibodies

In one aspect, the method of the invention involves treating a patient with Bordetella pertussis, comprising administering to the patient the modified humanized 1B7 antibody and/or the modified humanized 11E6 antibody, or pharmaceutical compositions including the antibody or antibodies.

In another aspect, the method of the invention involves a method of preventing a Bordetella pertussis infection, comprising administering to a patient the modified humanized 1B7 antibody and/or the modified humanized 11E6 antibody, or pharmaceutical compositions including the antibody or antibodies and, in some embodiments, the patient is at risk for a Bordetella pertussis infection (e.g. the patient is a pre-vaccination infant and/or the patient has been exposed to a pertussis toxin).

Leukocytosis or elevation in white blood cell count is characteristic of Bordetella pertussis infections. In one embodiment, the method of the invention comprises a reduction in white blood cell count in the patient. In an embodiment, the method of the invention results in an acceleration of the resolution of leukocytosis. In another embodiment, the method of the invention results in a reduction of the maximum white blood cell count during the course of the infection.

In various embodiments, the method of the invention results in an improvement of whooping cough in the patient. In one embodiment, the coughing symptoms of the patient are improved. For example, the method reduces the frequency of coughing or the number of coughs (or coughing episodes) in the patient. In various embodiments, the method reduces the number of coughs or coughing episodes by at least about 1 per hour, at least about 2 per hour, at least about 3 per hour, at least about 4 per hour, at least about 5 per hour, at least about 6 per hour, at least about 7 per hour, at least about 8 per hour, at least about 9 per hour, at least about 10 per hour, at least about 15 per hour, at least about 20 per hour, at least about 25 per hour, at least about 30 per hour, at least about 35 per hour, at least about 40 per hour, at least about 45 per hour, at least about 50 per hour, at least about 55 per hour, at least about 60 per hour, at least about 65 per hour, at least about 70 per hour, at least about 75 per hour, at least about 80 per hour, at least about 85 per hour, at least about 90 per hour, at least about 95 per hour, or at least about 100 per hour. In another example, the method reduces the duration of coughing in the patient. For example, the method reduces the duration of coughing during the course of the infection by at least about three months, about two months, about one month, about 4 weeks, about 3 weeks, about 2 weeks, about 1 week, about 5 days, about 4 days, about 3 days, about 2 days, or about 1 day. In a further embodiment, the number of whoops is reduced in the patient.

In another embodiment, the method of the invention reduces the level of the Bordetella pertussis bacteria in the nasopharynx of the patient. In a further embodiment, the method of the invention reduces the level of the Bordetella pertussis bacteria in the lung of the patient (e.g. bacterial lung colonization). For example, the method reduces the Bordetella pertussis levels in the nasopharynx and/or the lungs by about 95%, about 90%, about 80%, about 70%, about 60%, about 50%, about 40%, about 30%, about 20%, about 10%, or about 5%.

In one embodiment, the method of the invention results in neutralization (inhibition or antagonization) of the pertussis toxin protein. For example, antibodies of the invention can bind to the pertussis toxin protein so as to partially or completely inhibit one or more biological activities of the pertussis toxin protein. Among the biological activities of a pertussis toxin protein that a neutralizing antibody may inhibit or block is the ability of a pertussis toxin protein to bind cellular receptors. The receptor binding region of a pertussis toxin protein consists of four polypeptide subunits referred to as subunit S2, subunit S3, subunit S4 and subunit S5, respectively. Examples of cellular receptors that are bound by the subunits S2, S3, S4, and S5 of a pertussis toxin protein are members of the N-linked sialoglycoprotein family such as fetuin, haptoblobin, and transferrin. In an illustrative embodiment, the modified humanized antibodies of the invention prevent the pertussis toxin protein from binding to its cellular receptor. In another embodiment, the modified humanized antibodies of the invention alter the intracelluar trafficking steps of the pertussis toxin such that the toxin does not reach the cellular cytosol. Another important activity of a pertussis toxin protein that may be inhibited by antibodies of the invention is the enzymatic activity of the pertussis toxin protein as ADP ribosylase towards G proteins. The subunit conferring to the enzymatic activity as ADP-ribosylase in a pertussis toxin protein is subunit S1. In some embodiments, the pertussis toxin protein is a pertussis holotoxin. A pertussis holotoxin as referred to herein as a pertussis toxin protein that includes all five pertussis toxin protein subunits. In other embodiments, the pertussis toxin protein is a truncated pertussis toxin protein. A truncated pertussis protein as referred to herein includes at least one of the pertussis toxin protein subunits (i.e., S1, S2, S3, S4 and S5). Pertussis toxin proteins of various forms are described in, for example, U.S. Pat. No. 8,653,243, which is herein incorporated by reference in its entirety.

In various embodiments, the present compositions and methods are useful in the treatment or prevention of any of the stages of pertussis infections. For example, the incubation period of pertussis is commonly 7-10 days, with a range of 4-21 days, and rarely may be as long as 42 days. In various embodiments, the present compositions and methods increase the length of the incubation period by making the infection more difficult to come about. The clinical course of the illness is divided into three stages. The first stage, the catarrhal stage, is characterized by the insidious onset of coryza, sneezing, low-grade fever, and a mild, occasional cough, similar to the common cold. The cough gradually becomes more severe, and after 1-2 weeks, the second, or paroxysmal stage, begins. In various embodiments, the present compositions and methods, reduce the length of the catarrhal stage and, optionally, prevent it from advancing to the paroxysmal stage. In various embodiments, the present compositions and methods treat one or more of coryza, sneezing, low-grade fever, and cough. It is during the paroxysmal stage that the diagnosis of pertussis is usually suspected. Characteristically, a patient has bursts, or paroxysms, of numerous, rapid coughs, apparently due to difficulty expelling thick mucus from the tracheobronchial tree. At the end of the paroxysm, a long inspiratory effort is usually accompanied by a characteristic high-pitched whoop. During such an attack, the patient may become cyanotic. Children and young infants, especially, appear very ill and distressed. Vomiting and exhaustion commonly follow the episode. In various embodiments, the present compositions and methods reduce the quantity and/or frequency of paroxysms. In various embodiments, the present compositions and methods prevent a patient from becoming cyanotic. Paroxysmal attacks occur more frequently at night, with an average of 15 attacks per 24 hours. During the first 1 or 2 weeks of this stage, the attacks increase in frequency, remain at the same level for 2 to 3 weeks, and then gradually decrease. The paroxysmal stage usually lasts 1 to 6 weeks but may persist for up to 10 weeks. In various embodiments, the present compositions and methods reduce the length of this stage. In the convalescent stage, recovery is gradual. The cough becomes less paroxysmal and disappears in 2 to 3 weeks. In various embodiments, the present compositions and methods accelerate the onset of this stage and/or reduce its duration. Further, in various embodiments, the present compositions and methods prevent or reduce the recurrence of paroxysms, which may occur with subsequent respiratory infections. In various embodiments, the present compositions and methods prevent or reduce one or more of the onset of secondary bacterial pneumonia, neurologic complications such as seizures and encephalopathy, hypoxia, otitis media, dehydration, pneumothorax, epistaxis, subdural hematomas, hernias, rectal prolapsed, difficulty sleeping, urinary incontinence, pneumonia, and rib fracture. Further, in some embodiments, the present compositions and methods reduce or prevent necrotizing bronchiolitis, pneumonia (e.g. from Bordetella pertussis), pulmonary edema, pulmonary hypertension, and death.

In an embodiment, methods of the invention involve co-administration of the modified humanized 1B7 antibody and the modified humanized 11E6 antibody to the patient. In some embodiments, co-administration produces synergistic effects. Co-administration of the modified humanized 1B7 antibody and the modified humanized 11E6 antibody may be simultaneous or sequential.

In some embodiments, the modified humanized 1B7 antibody and the modified humanized 11E6 antibody are administered to a subject simultaneously. The term “simultaneously” as used herein, means that the modified humanized 1B7 antibody and the modified humanized 11E6 antibody are administered with a time separation of no more than about 60 minutes, such as no more than about 30 minutes, no more than about 20 minutes, no more than about 10 minutes, no more than about 5 minutes, or no more than about 1 minute. Administration of the modified humanized 1B7 antibody and the modified humanized 11E6 antibody can be by simultaneous administration of a single formulation (e.g., a formulation comprising the modified humanized 1B7 antibody and the modified humanized 11E6 antibody) or of separate formulations (e.g., a first formulation including the modified humanized 1B7 antibody and a second formulation including the modified humanized 11E6 antibody).

Co-administration does not require the therapeutic agents to be administered simultaneously, if the timing of their administration is such that the pharmacological activities of the modified humanized 1B7 antibody and the modified humanized 11E6 antibody overlap in time, thereby exerting a combined therapeutic effect. For example, the modified humanized 1B7 antibody and the modified humanized 11E6 antibody can be administered sequentially. The term “sequentially” as used herein means that the modified humanized 1B7 antibody and the modified humanized 11E6 antibody are administered with a time separation of more than about 60 minutes. For example, the time between the sequential administration of the modified humanized 1B7 antibody and the modified humanized 11E6 antibody can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the modified humanized 1B7 antibody and the modified humanized 11E6 antibody being administered.

Either the modified humanized 1B7 antibody or the modified humanized 11E6 antibody can be administered first. For example, the modified humanized 1B7 antibody can be administered to a subject after the time at which the modified humanized 11E6 antibody is administered. In this case, it is generally desirable to administer the modified humanized 1B7 antibody prior to the time at which about 50% (e.g., prior to the time at which about 40%, about 30%, about 20%, about 10%, or about 5%) of the modified humanized 11E6 antibody is metabolized or excreted by the subject, or the time at which the modified humanized 11E6 antibody has reached about 50%, about 60%, about 70%, about 80%, about 90%, or about 100% of its pharmacodynamic activity. In another example, the modified humanized 1B7 antibody can be administered to a subject before the administration of the modified humanized 11E6 antibody. In this case, it is generally desirable to administer the modified humanized 11E6 antibody prior to the time at which about 50% (e.g., prior to the time at which about 40%, about 30%, about 20%, about 10%, or about 5%) of the modified humanized 1B7 antibody is metabolized or excreted by the subject, or the time at which the modified humanized 1B7 antibody being administered has reached about 50%, about 60%, about 70%, about 80%, about 90%, or about 100% of its pharmacodynamic activity.

Co-administration also does not require the therapeutic agents to be administered to the patient by the same route of administration. Rather, each therapeutic agent can be administered by any appropriate route, for example, parenterally or non-parenterally. In an embodiment, the therapeutic agents may be administered orally to the subject. In another embodiment, the therapeutic agents may be administered parenterally, including for example, intravenous, intramuscular, intraperitoneal, subcutaneous and intra-articular injection and infusion, among others. In an embodiment, the therapeutic agents may be administered through intramuscular injection to the subject.

In some embodiments, the modified humanized antibodies of the present invention have a peak in a serum concentration (e.g. a beta half-life) of at least about 30, or about 35, or about 40, or about 45, or about 50, or about 55, or about 60, or about 65, or about 70, or about 75, or about 80 hours post-administration or at least about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, or about 25 days).

In some embodiments, the antibodies of the present invention have prolonged half-lives. In some embodiments, the antibodies of the present invention have an in vivo half-life of about 200, or about 225, or about 250, or about 275, or about 300 hours or about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, or about 50 days, e.g. about 1 or about 2 weeks, or about 3 weeks, about 4 weeks, about 5 weeks, about 6 weeks, about 7 weeks, or about 8 weeks). In an embodiment, the antibodies of the present invention have in vivo half-life of about 20 days to about 40 days.

Accordingly, in some embodiments, a patient may receive a first administration (e.g. infusion or intramuscular (IM) injection) of the inventive antibodies as part of a treatment method and may receive a further administration (e.g. infusion or intramuscular injection) after a peak in serum concentration and/or the in vivo half-life of the antibodies of the present invention (e.g. the dose of the further administration may be identical to the first administration or may be lower, e.g. a maintenance dose). In some embodiments, the further administration is about one day from the first administration, or about one week from the first administration. In some embodiments, the present methods provide for about 1-3 (e.g. about 1, or about 2, or about 3) doses (e.g. IV doses or IM doses) of the antibodies of the present invention per week (or about every 5, or 6, or 7, or 10 days). In some embodiments, the present methods maintain a therapeutic window of antibody levels in the blood serum of about 5 μg/mL, about 10 μg/mL, about 20 μg/mL, about 25 μg/mL, about 50 μg/mL, about 75 μg/mL, or about 100 μg/mL, or about 125 μg/mL, or about 150 μg/mL, or about 175 μg/mL, or about 200 μg/mL, or about 225 μg/mL, or about 250 μg/mL, or about 300 μg/mL. In some embodiments, the present methods allow for infrequent dosing and/or lower dosing (e.g. longer half-lives permitting lower and less frequent dosing).

In another embodiment, the method includes administering to a patient the modified humanized 1B7 antibody and/or the modified humanized 11E6 antibody, along with antimicrobial agents. It is contemplated that co-administration of the modified humanized 1B7 antibody and/or the modified humanized 11E6 antibody along with antimicrobial agents produces synergistic effects. Illustrative antimicrobial agents that may be used for the invention include, but are not limited to azithromycin, clarithromycin, erythromycin, trimethoprim-sulfamethoxasole, roxithromycin, ketolides (e.g., telithromycin) ampicillin, amoxicillin, tetracycline, chloramphenicol, fluoroquinolones (e.g., ciprofloxacin, levofloxacin, ofloxacin, moxifloxacin), and cephalosporins. In an embodiment, the antimicrobial agent is erythromycin.

In various embodiments, the method of the invention treats human patients. In an embodiment, the human patient is an infant. In an embodiment, the human patient is a newborn. In another embodiment, the human patient is a neonate who is less than four weeks old, less than three weeks old, less than two weeks old, less than one week old, less than six days old, less than five days old, less than four days old, less than three days old, less than two days old, or less than one day old. In some embodiments, the human is one month old, two months old, three months old, four months old, five months old, or six months old. In some embodiments, the human has an age in a range of from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In a further aspect, the method of the invention prevents Bordetella pertussis infection in a subject previously exposed to the bacteria, comprising administering to the subject the modified humanized 1B7 antibody and/or the modified humanized 11E6 antibody, or pharmaceutical compositions including the antibody or antibodies. In various embodiments, the method provides an effective prophylactic treatment in preventing Bordetella pertussis infection in a subject exposed to the bacteria.

In various embodiments, the antibodies of the invention exhibit extended half-life, and based on the exponential nature of half-life, small changes to the length of half-life can cause substantial improvements to the duration of prophylaxis. Accordingly, in various embodiments, the antibodies of the invention provide a prophylaxis time (i.e., the time that the subject is protected from Bordetella pertussis infection) of at least one day, at least two days, at least three days, at least four days, at least five days, at least six days, at least 1 week, at least 2 weeks, at least 3 weeks, at least 4 weeks, at least 1 month, at least 2 months, at least 3 months, at least 4 months, at least 5 months, at least 6 months, or at least 1 year.

In some embodiments, the antibody of the invention (e.g., modified humanized 1B7 antibody and/or modified humanized 11E6 antibody) is utilized in prophylactic applications in a subject who has not been previously vaccinated against the bacteria. In an embodiment, the antibody of the invention is administered to a subject as a prophylactic treatment prior to the subject receiving a pertussis vaccination. In various embodiments, the antibody of the invention is utilized in prophylactic treatments of a subject who is less than one year old, less than eleven months old, less than ten months old, less than nine months old, less than eight months old, less than seven months old, less than six months old, less than five months old, less than four months old, less than three months old, less than two months old, less than one month old, less than four weeks old, less than three weeks old, less than two weeks old, less than one week old, less than six days old, less than five days old, less than four days old, less than three days old, less than two days old, or less than one day old. Accordingly, in some embodiments, the present methods involving bridging the time between birth and vaccination in an infant patient.

In various embodiments, the methods of the invention treat or prevent Bordetella pertussis infection in a subject previously vaccinated against the bacteria. In an embodiment, the subject is an infant or child vaccinated with DtaP (e.g., INFANRIX (with three antigens, mostly pertussis toxin (PT) and FHA), TRIPEDIA (which contains two components, FHA and PT, in equal amounts) and DAPTACEL (which contains five components, PT, FHA, pertactin, and fimbriae types 2 and 3)). In another embodiment, the subject is an adult vaccinated with the pertussis booster vaccine Tdap (e.g. BOOSTRIX (with three pertussis antigens (PT, FHA, and pertactin) in a reduced quantity compared with INFANRIX) and ADACEL (with the same five pertussis components as DAPTACEL but with a reduced quantity of PT). In other embodiments, the patient of the present invention may or may not have received any one of the following pertussis combination vaccines: PEDIARIX, PENTACEL, or KINRIX.

It is contemplated that the humanized antibodies of the invention may further function as adjuvant for vaccinations such as DtaP or Tdap. Further, in various embodiments, the methods of the invention treat or prevent Bordetella pertussis infection in a subject that has not been previously vaccinated against the bacteria

In various embodiments, the present compositions and methods supplement or supplant treatment with palivizumab (SYNAGIS).

In various embodiments, the present compositions and methods can treat pertussis infections that have various strains as their etiology, including, by way of non-limiting example, pertactin-negative pertussis.

Furthermore, Bordetella parapertussis is a closely related species Bordetella pertussis. Both bacteria are linked to outbreaks of whooping cough in humans and produce similar virulence factors. Co-infection of Bordetella pertussis and Bordetella parapertussis is not unusual. Accordingly, in one aspect of the invention, the method of the invention involves treating a patient with Bordetella parapertussis, comprising administering to the patient the modified humanized 1B7 antibody and/or the modified humanized 11E6 antibody, or pharmaceutical compositions including the antibody or antibodies. In another aspect of the invention, the method of the invention prevents Bordetella parapertussis infection in a subject previously exposed to the bacteria, comprising administering to the subject the modified humanized 1B7 antibody and/or the modified humanized 11E6 antibody, or pharmaceutical compositions including the antibody or antibodies.

In various embodiments, the methods of the invention are effective in treating Bordetella pertussis infection and/or Bordetella parapertussis infection when the modified humanized 1B7 antibody and/or the modified humanized 11E6 antibody is administered to the patient at about 3 months after infection. In other embodiments, the methods of the invention are effective in treating Bordetella pertussis infection and/or Bordetella parapertussis infection when the modified humanized 1B7 antibody and/or the modified humanized 11E6 antibody is administered to the patient at about 2 months, about 1 month, about 4 weeks, about 3 weeks, about 2 weeks, about 7 days, about 6 days, about 5 days, about 4 days, about 3 days, about 2 days, or about 1 day after infection. In an embodiment, the modified humanized 1B7 antibody and/or the modified humanized 11E6 antibody is administered to the patient on the day of infection.

As used herein, “treat,” “treating” and “treatment” mean the treatment of a disease in a mammal, e.g., in a human. In various embodiments, this includes: (a) inhibiting the disease, i.e., arresting its development and/or (b) relieving the disease, i.e., causing regression of the disease state.

### Pharmaceutical Compositions and Administration

The pharmaceutical compositions of the invention can be administered for therapeutic or prophylactic treatment. For such uses, an antibody preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing antibodies, such as those disclosed herein, can be presented in a dosage unit form and can be prepared by any suitable method. A pharmaceutical composition should be formulated to be compatible with its intended route of administration. Examples of routes of administration are oral, intranasal, pulmonary, intravenous (IV), intradermal, inhalation, transdermal, topical, transmucosal, subcutaneous, intramuscular (IM), intraperitoneal, and rectal administration. In an embodiment, the route of administration for antibodies of the invention is IV infusion. In another embodiment, the route of administration for antibodies of the invention is IM injection.

Useful formulations can be prepared by methods well known in the pharmaceutical art. For example, pharmaceutical compositions of the invention can be formulated as a colloidal dispersion system, macromolecular complex, nanocapsule, microsphere, bead, oil-in-water emulsion, micelle, mixed micelle, or liposome. For example, see Remington's Pharmaceutical Sciences, 18th ed. (Mack Publishing Company, 1990).

Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfate; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can also contain other compatible therapeutic agents. For example, the composition may additionally include antimicrobial agents described herein.

The combined administrations contemplates co-administration, using separate formulations or a single pharmaceutical formulation, and consecutive administration in either order, wherein preferably there is a time period while both (or all) active agents simultaneously exert their biological activities. In an embodiment, a pharmaceutical composition of the invention includes a formulation of the modified humanized 1B7 antibody. In another embodiment, a pharmaceutical composition of the invention includes a formulation of the modified humanized 11E6 antibody. In a further embodiment, a pharmaceutical composition of the invention includes a co-formulation of both the modified humanized 1B7 antibody and the modified humanized 11E6 antibody.

It will be appreciated that the actual dose of the antibodies (e.g., modified humanized 1B7 antibody and/or modified humanized 11E6 antibody) to be administered according to the present invention will vary according to, for example, the particular dosage form and the mode of administration. Many factors that may modify the action of the antibodies (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

Individual doses of the antibody (e.g., modified humanized 1B7 antibody and/or modified humanized 11E6 antibody) can be administered in unit dosage forms containing, for example, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg active ingredient, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg per unit dosage form, or from about 5 mg to about 80 mg per unit dosage form. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, inclusive of all values and ranges therebetween.

In one embodiment, the antibody (e.g., modified humanized 1B7 antibody and/or modified humanized 11E6 antibody) is administered at an amount of from about 0.01 mg to about 100 mg daily, an amount of from about 0.01 mg to about 1,000 mg daily from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various embodiments, the antibody is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, inclusive of all values and ranges therebetween.

In some embodiments, a suitable dosage of the antibody (e.g., modified humanized 1B7 antibody and/or modified humanized 11E6 antibody) is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, or about 100 mg/kg body weight, inclusive of all values and ranges therebetween. In other embodiments, a suitable dosage of the antibody in a range of about 0.01 mg/kg to about 100 mg/kg of body weight, in a range of about 1 mg/kg to about 100 mg/kg of body weight, in a range of about 1 mg/kg to about 90 mg/kg of body weight, in a range of about 1 mg/kg to about 80 mg/kg of body weight, in a range of about 1 mg/kg to about 70 mg/kg of body weight, in a range of 1 mg/kg to about 60 mg/kg of body weight, in a range of 1 mg/kg to about 50 mg/kg of body weight, in a range of 1 mg/kg to about 40 mg/kg of body weight, in a range of 1 mg/kg to about 30 mg/kg of body weight, in a range of 1 mg/kg to about 20 mg/kg of body weight, in a range of about 5 mg/kg to about 50 mg/kg of body weight, in a range of about 5 mg/kg to about 40 mg/kg of body weight, in a range of about 5 mg/kg to about 30 mg/kg of body weight, in a range of about 5 mg/kg to about 20 mg/kg of body weight, inclusive of all values and ranges therebetween.

In accordance with certain embodiments of the invention, the antibody (e.g., modified humanized 1B7 antibody and/or modified humanized 11E6 antibody) may be administered, for example, more than once daily, about once per day, about every other day, about every third day, about once a week, about once every two weeks, about once every month, about once every two months, about once every three months, about once every six months, or about once every year.

Antibody can be administered on multiple occasions. Intervals between single dosages can be weekly, monthly or yearly. Intervals can also be irregular as indicated by measuring blood levels of the antibody in the subject. In some embodiments, the antibody can be administered as a sustained release formulation, in which case less frequent administration is required.

In some methods, the antibody of the invention is administered at a dosage to achieve a plasma or serum antibody concentration of 1-1000 μg/ml and in some methods 25-300 μg/ml. For example, the antibody of the invention can be administered at a dosage to achieve a plasma or serum level of about 1-1000 μg/ml, 1-900 μg/ml, 1-800 μg/ml, 1-700 μg/ml, 1-600 μg/ml, 1-500 μg/ml, 1-400 μg/ml, 1-300 μg/ml, 1-200 μg/ml, 1-100 μg/ml, 10-500 μg/ml, 10-400 μg/ml, 10-300 μg/ml, 10-200 μg/ml, 10-100 μg/ml, 100-400 μg/ml, 100-300 μg/ml, or 100-200 μg/ml, inclusive of all values and ranges therebetween. For example, the antibody of the invention can be administered at a dosage to achieve a plasma or serum level of about 1 μg/ml, about 5 μg/ml, about 10 μg/ml, about 15 μg/ml, about 20 μg/ml, about 25 μg/ml, about 30 μg/ml, about 35 μg/ml, about 40 μg/ml, about 45 μg/ml, about 50 μg/ml, about 55 μg/ml, about 60 μg/ml, about 65 μg/ml, about 70 μg/ml, about 75 μg/ml, about 80 μg/ml, about 85 μg/ml, about 90 μg/ml, about 95 μg/ml, about 100 μg/ml, about 105 μg/ml, about 110 μg/ml, about 115 μg/ml, about 120 mg μg/ml, about 125 μg/ml, about 130 μg/ml, about 135 μg/ml, about 140 μg/ml, about 145 μg/ml, about 150 μg/ml, about 155 μg/ml, about 160 μg/ml, about 165 μg/ml, about 170 μg/ml, about 175 μg/ml, about 180 μg/ml, about 185 μg/ml, about 190 μg/ml, about 195 μg/ml, about 200 μg/ml, about 205 μg/ml, about 210 μg/ml, about 215 μg/ml, about 220 mg μg/ml, about 225 μg/ml, about 230 μg/ml, about 235 μg/ml, about 240 μg/ml, about 245 μg/ml, about 250 μg/ml, about 255 μg/ml, about 260 μg/ml, about 265 μg/ml, about 270 μg/ml, about 275 μg/ml, about 280 μg/ml, about 285 μg/ml, about 290 μg/ml, about 295 μg/ml, or about 300 μg/ml.

In some methods, the antibody of the invention (e.g., modified humanized 1B7 antibody and/or modified humanized 11E6 antibody) achieves a potency of at least about 1 EU/μg, at least about 2 EU/ug, at least about 3 EU/ug, at least about 4 EU/ug, at least about 5 EU/ug, at least about 6 EU/ug, at least about 7 EU/ug, at least about 8 EU/ug, at least about 9 EU/ug, at least about 10 EU/ug, at least about 15 EU/ug, at least about 20 EU/ug, at least about 25 EU/ug, at least about 30 EU/ug, at least about 35 EU/ug, at least about 40 EU/ug, at least about 45 EU/ug, at least about 50 EU/ug, at least about 55 EU/ug, at least about 60 EU/ug, at least about 65 EU/ug, at least about 70 EU/ug, at least about 75 EU/ug, at least about 80 EU/ug, at least about 85 EU/ug, at least about 90 EU/ug, at least about 95 EU/ug, at least or about 100 EU/ug. In some methods, the antibody of the invention (e.g., modified humanized 1B7 antibody and/or modified humanized 11E6 antibody) achieves a potency of at least about 1 EU/ml, at least about 2 EU/ml, at least about 3 EU/ml, at least about 4 EU/ml, at least about 5 EU/ml, at least about 6 EU/ml, at least about 7 EU/ml, at least about 8 EU/ml, at least about 9 EU/ml, at least about 10 EU/ml, at least about 15 EU/ml, at least about 20 EU/ml, at least about 25 EU/ml, at least about 30 EU/ml, at least about 35 EU/ml, at least about 40 EU/ml, at least about 45 EU/ml, at least about 50 EU/ml, at least about 55 EU/ml, at least about 60 EU/ml, at least about 65 EU/ml, at least about 70 EU/ml, at least about 75 EU/ml, at least about 80 EU/ml, at least about 85 EU/ml, at least about 90 EU/ml, at least about 95 EU/ml, at least or about 100 EU/ml. EU stands for equivalent units as defined by the WHO polyclonal serum standard. In various embodiments, the antibody of the invention is able to maintain potency after at least about 1 day, about 2 days, about 3 days, about 4 days, about 5 days, about 6 days, about 1 week, about 2 weeks, about 3 weeks, about 4 weeks, about 5 months, about 6 months, about 7 months, about 8 months, about 9 months, about 10 months, about 11 months, or about 12 months.

Dosage and frequency vary depending on factors such as route of administration, dosage amount, the disease being treated, and the half-life of the antibody in the patient. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime. Illustrative dosing frequencies are once per day, twice per day, three times per day, once per week and once every two weeks. In some embodiments, dosing is once every two weeks.

The invention also provides kits that can simplify the administration of any agent described herein (e.g. the humanized antibodies with or without various combination agents). An illustrative kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

In some embodiments, the kit ma comprises a pre-filled syringe in unit dose form (e.g. an injector pen). In various embodiments, the kits are suited for use away from a traditional medical center, e.g. in the field, e.g. in the third world

### EXAMPLES

### Example 1: Generation and In Vitro Characterization of the hu1B7-YTE Extended Half-Life Antibody

To extend the plasma half-life of the hu1B7 antibody, three amino acid changes were introduced into the CH₂ domain of the human IgG1 Fc region (Dall'Acqua, Kiener et al. 2006, Robbie, Criste et al. 2013). These changes, M252Y/S254T/T256E, referred to as YTE, function to increase the antibody's affinity for the neonatal Fc receptor, but only at the low, endosomal pH. Thus, when the antibody is taken up by this receptor, it is not released in the endosome and is recycled to the cell surface where it is released back into circulation. The YTE changes may also reduce binding to several Fcγ receptors, in particular FcγRIII (Ko, Pegu et al. 2014, Grevys, Bern et al. 2015).

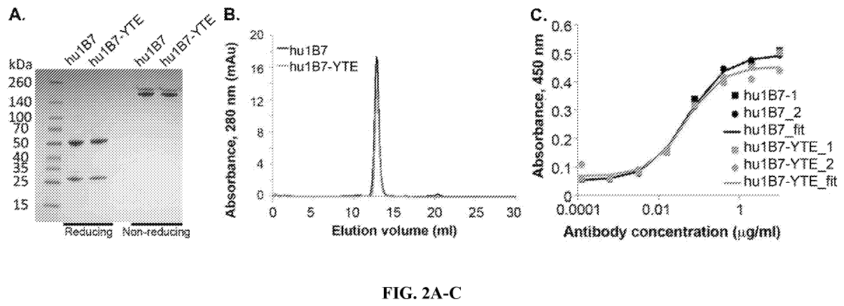

Hu1B7-YTE extended half-life antibody was generated via transient expression in CHO cells. The antibody expressed at the same level as the parent antibody (2.3 pg/cell/day). The antibody was subjected to Protein A purification, and SDS-PAGE analyses revealed that no degradation products or high molecular weight aggregates were observed (FIG. 2, panel A). Hu1B7 and hu1B7-YTE were compared via SDS PAGE analyses. Under reducing conditions, both hu1B7 and hu1B7-YTE displayed 50 kDa heavy chain and 25 kDa light chain bands (FIG. 2, panel A). Under non-reducing SDS PAGE conditions a single band of 150 kDa was seen with both antibodies (FIG. 2, panel A). Size exclusion chromatography using a Superdex 5200 column verified that both antibodies were predominantly monomeric with >97% eluted as a single peak corresponding to a molecular weight of −150 kDa (FIG. 2, panel B).

ELISA analyses were performed to compare the affinity to pertussis toxin of hu1B7-YTE and hu1B7. The ELISA plates were coated with pertussis toxin, two duplicate samples of each purified antibody were titrated from 3 μg/ml, and the bound antibodies were detected with goat-anti-human-kappa-constant-domain-HRP. The ELISA assays verified that hu1B7-YTE bound pertussis toxin as effectively as the original hu1B7 antibody (FIG. 2, panel C).

Altogether, these data demonstrated that the hu1B7-YTE antibody was biochemically identical to the original hu1B7 antibody.

### Example 2: In Vivo Characterization of the hu1B7 and hu1B7-YTE Antibodies in Pertussis Prophylaxis

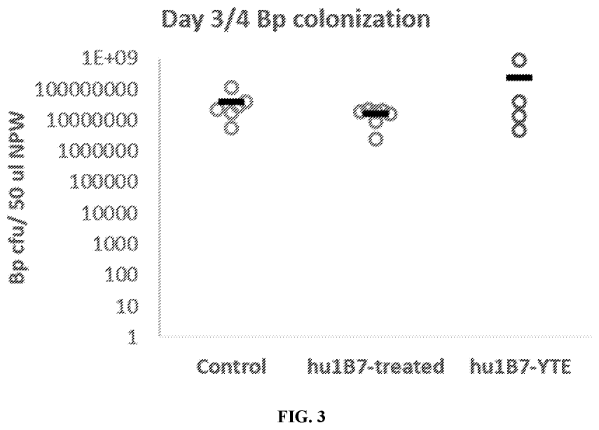

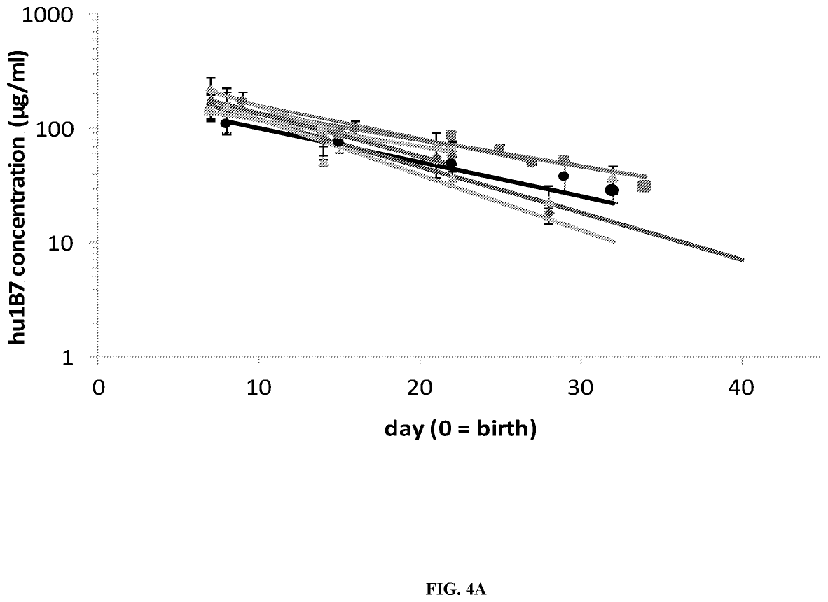

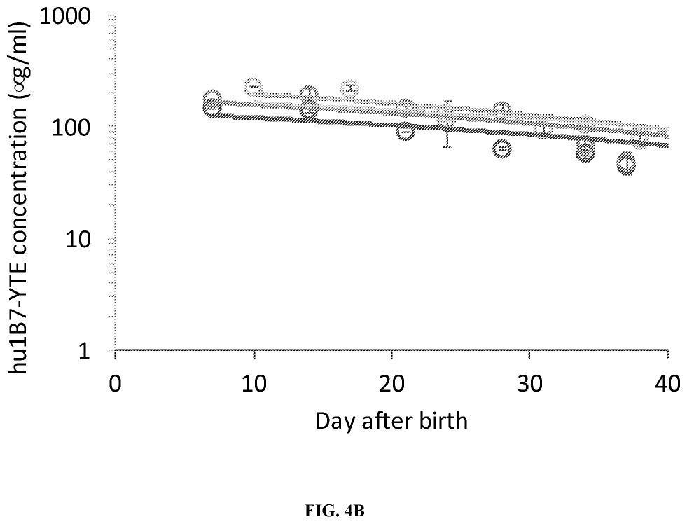

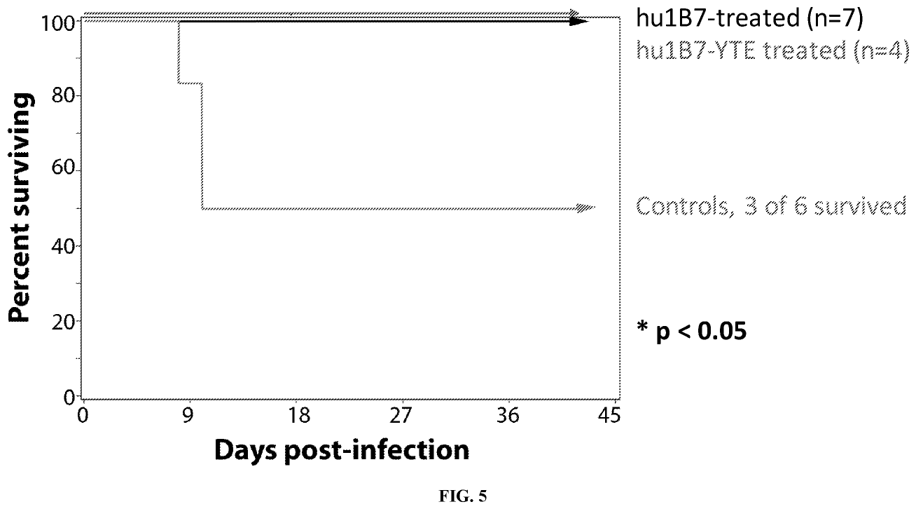

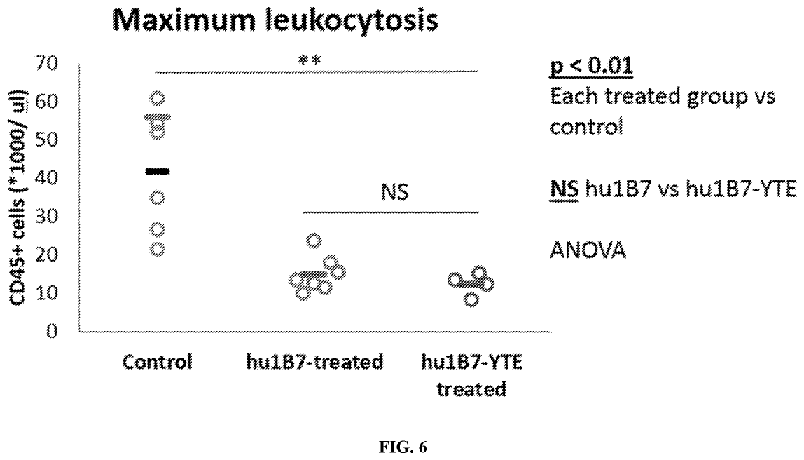

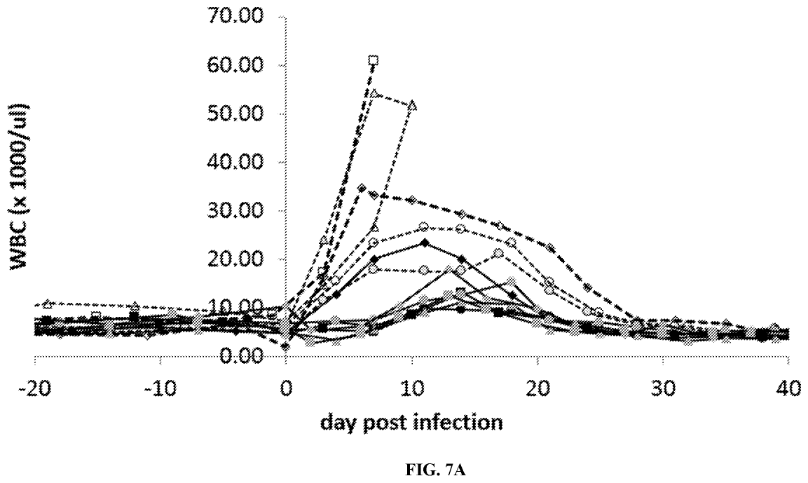

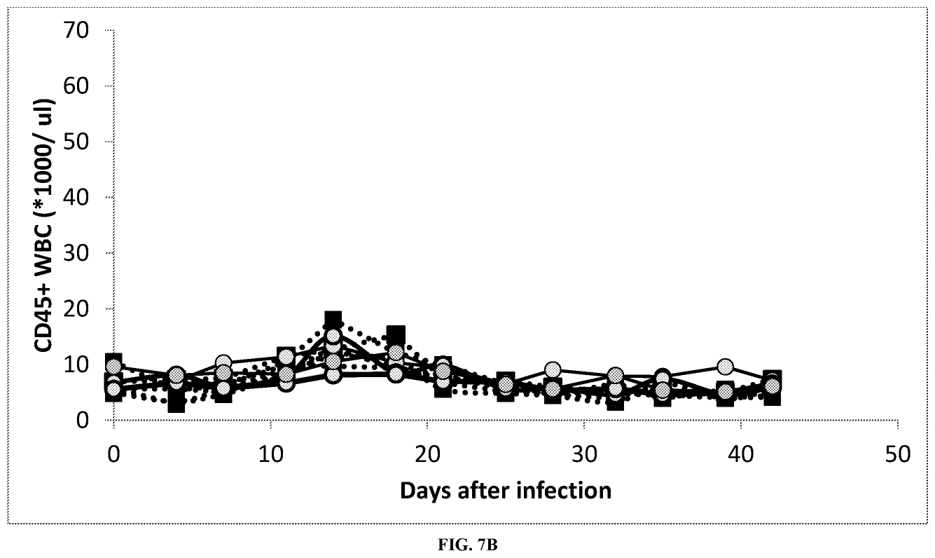

To evaluate the potential of hu1B7 and hu1B7-YTE to provide pertussis prophylaxis in newborn baboons, two day old baboons were injected with hu1B7 (8 treated animals) or hu1B7-YTE (7 treated animals, with data available and presented for 4), while control animals were untreated (7 animals, with data available and presented for 6). Animals treated with the antibodies each received 40 mg/kg of the indicated antibodies intravenously (IV) at day 2. Serum levels of the hu1B7 and hu1B7-YTE were followed for 5 weeks, at which time the animals were infected with 10⁸ cfu of B. pertussis strain D420 via intra-tracheal and intranasal infusions. The animals were monitored for clinical signs of disease, including leukocytosis, coughing, and bacterial colonization. The pharmacokinetics of hu1B7 and hu1B7-YTE were assessed and the hu1B7 and hu1B7-YTE half-lives were calculated. All animals were heavily colonized during the first week after infection as measured by B. pertussis in the nasopharyngeal wash (NPW) fluid (FIG. 3). Elimination kinetics were calculated for hu1B7-treated (FIG. 4A) and hu1B7-YTE-treated (FIG. 4B) neonatal baboons. Results of these studies are shown in Table 1 and demonstrate that hu1B7-YTE has an extended half-life in neonatal baboons compared to hu1B7. Furthermore, hu1B7-treated and hu1B7-YTE-treated neonatal baboons demonstrated enhanced survival relative to control animals (FIG. 5). Both hu1B7-treated and hu1B7-YTE-treated animals exhibited reduced maximum leukocytosis (FIG. 6). Likewise, both hu1B7-treated and hu1B7-YTE-treated animals showed a smaller and delayed increase in WBC following infection as compared to control animals (FIG. 7).

TABLE 1
In vivo stability of hu1B7 and hu1B7-YTE antibodies

| | | |
|---|---|---|
| hu1B7-YTE | hu1B7 | P-value |

| | | | | | |
|---|---|---|---|---|---|
| t_{1/2} b, mean | 23 ± 6 | days | 11.8 ± 4 | days | 0.01 |
| t_{1/2} b, range | 19-32 | days | 7-14 | days | |
| Ave hu1B7 | 84 ± 25 | ug/ml | 35 ± 18 | ug/ml | 0.01 |
| concentration | | | | | |
| at infection | | | | | |

These data demonstrate that monoclonal antibody prophylaxis of newborn baboons with hu1B7 and hu1B7-YTE mitigated the clinical signs of pertussis, including leukocytosis and coughing, but did not prevent bacterial colonization. The elimination half-life of hu1B7 of −12 days was the expected half-life of a humanized monoclonal antibody in a non-human primate. Hu1B7-YTE exhibited an increased half-life of −23 days while retaining therapeutic efficacy, which could offer an increased period of protection. These results provide proof-of-concept that passive immunization with hu1B7 and hu1B7-YTE can protect infants from pertussis in the developing world.

### INCORPORATION BY REFERENCE

The entire disclosure of each of the patent documents and scientific articles referred to herein is incorporated by reference for all purposes.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections

### EQUIVALENTS

The invention may be embodied in other specific forms without departing from the spirit or essential characteristics thereof. The foregoing embodiments are therefore to be considered in all respects illustrative rather than limiting on the invention described herein.

### REFERENCES

The entire disclosure of each of following references are incorporated by reference for all purposes:
1. Dall'Acqua, W. F., P. A. Kiener and H. Wu (2006). “Properties of human IgGls engineered for enhanced binding to the neonatal Fc receptor (FcRn).” J Biol Chem 281(33): 23514-23524.
2. Grevys, A., M. Bern, S. Foss, D. B. Bratlie, A. Moen, K. S. Gunnarsen, A. Aase, T. E. Michaelsen, I. Sandlie and J. T. Andersen (2015). “Fc Engineering of Human IgG1 for Altered Binding to the Neonatal Fc Receptor Affects Fc Effector Functions.” J Immunol 194(11): 5497-5508.
3. Ko, S. Y., A. Pegu, R. S. Rudicell, Z. Y. Yang, M. G. Joyce, X. Chen, K. Wang, S. Bao, T. D. Kraemer, T. Rath, M. Zeng, S. D. Schmidt, J. P. Todd, S. R. Penzak, K. O. Saunders, M. C. Nason, A. T. Haase, S. S. Rao, R. S. Blumberg, J. R. Mascola and G. J. Nabel (2014). “Enhanced neonatal Fc receptor function improves protection against primate SHIV infection.” Nature 514(7524): 642-645.
4. Robbie, G. J., R. Criste, W. F. Dall′acqua, K. Jensen, N. K. Patel, G. A. Losonsky and M. P. Griffin (2013). “A novel investigational Fc-modified humanized monoclonal antibody, motavizumab-YTE, has an extended half-life in healthy adults.” Antimicrob Agents Chemother 57(12): 6147-615.

What is claimed is:

1. A humanized 1B7 antibody that binds a pertussis toxin protein, comprising an immunoglobulin heavy chain variable region, an immunoglobulin light chain variable region, and a modified human IgG1 constant region or fragment thereof, wherein
the immunoglobulin heavy chain variable region comprises a CDR_{H1} comprising an amino acid sequence selected from SEQ ID NO:26, a CDR_{H2} comprising an amino acid sequence selected from SEQ ID NO:27 and SEQ ID NO:28, and/or a CDR_{H3} comprising an amino acid sequence selected from SEQ ID NO:29;
the immunoglobulin light chain variable region comprises a CDR_{L1} comprising an amino acid sequence selected from SEQ ID NO:30, SEQ ID NO:31, and SEQ ID NO:32, a CDR_{L2} comprising an amino acid sequence selected from SEQ ID NO:33 and SEQ ID NO:34, and/or a CDR_{L3} comprising an amino acid sequence selected from SEQ ID NO:35; and
the modified human IgG1 constant region or fragment thereof comprises one or more amino acid substitutions relative to a wild-type human IgG1 constant region at one or more of amino acid residues 252, 254, 256, 433, 434, or 436, numbered according to the EU index as in Kabat.

2. The humanized 1B7 antibody of claim 1, wherein
the immunoglobulin heavy chain variable region comprises an amino acid sequence selected from SEQ ID NO:1, SEQ ID NO:2, SEQ ID NO:3, SEQ ID NO:4, SEQ ID NO:5, and SEQ ID NO:6; and
the immunoglobulin light chain variable region comprises an amino acid sequence selected from SEQ ID NO:7, SEQ ID NO:8, SEQ ID NO:9, SEQ ID NO:10, SEQ ID NO:11, and SEQ ID NO:12.

3. The humanized 1B7 antibody of claim 1 or 2, comprising an immunoglobulin heavy chain variable region and an immunoglobulin light chain variable region selected from:
(a) an immunoglobulin heavy chain variable region comprising the amino acid sequence of SEQ ID NO: 2, and an immunoglobulin light chain variable region comprising the amino acid sequence of SEQ ID NO:8;
(b) an immunoglobulin heavy chain variable region comprising the amino acid sequence of SEQ ID NO: 3, and an immunoglobulin light chain variable region comprising the amino acid sequence of SEQ ID NO:9;
(c) an immunoglobulin heavy chain variable region comprising the amino acid sequence of SEQ ID NO: 4, and an immunoglobulin light chain variable region comprising the amino acid sequence of SEQ ID NO:10;
(d) an immunoglobulin heavy chain variable region comprising the amino acid sequence of SEQ ID NO: 5, and an immunoglobulin light chain variable region comprising the amino acid sequence of SEQ ID NO:11; and
(e) an immunoglobulin heavy chain variable region comprising the amino acid sequence of SEQ ID NO: 6, and an immunoglobulin light chain variable region comprising the amino acid sequence of SEQ ID NO:12.

4. A humanized 11E6 antibody that binds a pertussis toxin protein, comprising an immunoglobulin heavy chain variable region, an immunoglobulin light chain variable region, and a modified human IgG1 constant region or fragment thereof, wherein
the immunoglobulin heavy chain variable region comprises a CDR_{H1} comprising an amino acid sequence selected from SEQ ID NO:36, a CDR_{H2} comprising an amino acid sequence selected from SEQ ID NO:37 and SEQ ID NO:38, and/or a CDR_{H3} comprising an amino acid sequence selected from SEQ ID NO:39;
the immunoglobulin light chain variable region comprises a CDR_{L1} comprising an amino acid sequence selected from SEQ ID NO:40, a CDR_{L2} comprising an amino acid sequence selected from SEQ ID NO:41, and/or a CDR_{L3} comprising an amino acid sequence selected from SEQ ID NO:42; and
the modified human IgG1 constant region or fragment thereof comprises one or more amino acid substitutions relative to a wild-type human IgG constant region at one or more of amino acid residues 252, 254, 256, 433, 434, or 436, numbered according to the EU index as in Kabat.

5. The humanized 11E6 antibody of claim 4, wherein
the immunoglobulin heavy chain variable region comprises an amino acid sequence selected from SEQ ID NO:13, SEQ ID NO:14, SEQ ID NO:15, SEQ ID NO:16, SEQ ID NO:17, and SEQ ID NO:18; and
the immunoglobulin light chain variable region comprises an amino acid sequence selected from SEQ ID NO:19, SEQ ID NO:20, SEQ ID NO:21, SEQ ID NO:22, SEQ ID NO:23, and SEQ ID NO:24;

6. The humanized 11E6 antibody of claim 4 or 5, comprising an immunoglobulin heavy chain variable region and an immunoglobulin light chain variable region selected from:
(a) an immunoglobulin heavy chain variable region comprising the amino acid sequence of SEQ ID NO: 14, and an immunoglobulin light chain variable region comprising the amino acid sequence of SEQ ID NO:20;
(b) an immunoglobulin heavy chain variable region comprising the amino acid sequence of SEQ ID NO: 15, and an immunoglobulin light chain variable region comprising the amino acid sequence of SEQ ID NO:21;
(c) an immunoglobulin heavy chain variable region comprising the amino acid sequence of SEQ ID NO: 16, and an immunoglobulin light chain variable region comprising the amino acid sequence of SEQ ID NO:22;
(d) an immunoglobulin heavy chain variable region comprising the amino acid sequence of SEQ ID NO:17, and an immunoglobulin light chain variable region comprising the amino acid sequence of SEQ ID NO:23; and
(e) an immunoglobulin heavy chain variable region comprising the amino acid sequence of SEQ ID NO:18, and an immunoglobulin light chain variable region comprising the amino acid sequence of SEQ ID NO:24.

7. The antibody of any one of claims 1-3, wherein the antibody binds the pertussis toxin protein with a K_{D} of 3 nM or lower.

8. The antibody of any one of claims 4-6, wherein the antibody binds the pertussis toxin protein with a K_{D} of 12 nM or lower.

9. A humanized 1B7 antibody that binds a pertussis toxin protein, wherein the antibody binds the pertussis toxin protein with a K_{D} of 3 nM or lower.

10. The antibody of claim 9, wherein the K_{D} is about 3 nM, or about 2 nM, or about 1 nM, or about 0.5 nM.

11. A humanized 11E6 antibody that binds a pertussis toxin protein, wherein the antibody binds the pertussis toxin protein with a K_{D} of 12 nM or lower.

12. The antibody of claim 11, wherein the K_{D} is about 12 nM, or about 10 nM, or about 8 nM, or about 6 nM, or 4 nM, or 2 nM, or about 1 nM, or about 0.5 nM.

13. The antibody of any one of claims 1-12, wherein the modified human IgG1 constant region or fragment thereof exhibits increased affinity for the neonatal Fc receptor (FcRn) relative to the wild-type human IgG1 constant region.

14. The antibody of any one of claims 1-13, wherein the modified human IgG1 constant region or fragment thereof comprises a triple M252Y/S254T/T256E mutation.

15. The antibody of any one of claims 1-13, wherein the modified human IgG1 constant region or fragment thereof comprises a triple H433K/N434F/Y436H mutation.

16. The antibody of any one of claims 1-13, wherein the modified human IgG1 constant region or fragment thereof comprises a triple M252Y/S254T/T256E mutation and a triple H433K/N434F/Y436H mutation.

17. An isolated nucleic acid comprising a nucleotide sequence encoding an immunoglobulin heavy chain variable region of any one of claims 1-16.

18. An isolated nucleic acid comprising a nucleotide sequence encoding an immunoglobulin light chain variable region of any one of claims 1-16.

19. An isolated nucleic acid comprising a nucleotide sequence encoding a human IgG1 constant region or fragment thereof of any one of claims 1-16.

20. An expression vector comprising the nucleic acid of any one of claims 17-19.

21. A host cell comprising the expression vector of claim 20.

22. A method of producing a polypeptide comprising an immunoglobulin heavy chain variable region, an immunoglobulin light chain variable region, or an human IgG1 constant region or fragment thereof, the method comprising:
(a) growing the host cell of claim 21 under conditions so that the host cell express the polypeptide comprising the immunoglobulin heavy chain variable region, the immunoglobulin light chain variable region, or the human IgG1 constant region or fragment thereof; and
(b) purifying the polypeptide comprising the immunoglobulin heavy chain variable region, the immunoglobulin light chain variable region, or the human IgG1 constant region or fragment thereof.

23. A method of producing an antibody that binds a pertussis toxin protein, the method comprising:
(a) growing the host cell of claim 21 under conditions so that the host cell expresses a polypeptide comprising the immunoglobulin heavy chain variable region and/or the immunoglobulin light chain variable region and/or the human IgG1 constant region or fragment thereof, thereby producing the antibody; and
(b) purifying the antibody.

24. A pharmaceutical composition comprising one or more antibodies of any one of claims 1-16, and a pharmaceutically acceptable excipient.

25. The pharmaceutical composition of claim 24, comprising the humanized 1B7 antibody of any one of claim 1-3, 7, 9, or 10, and the humanized 11E6 antibody of any one of claim 4-6, 8, 11, or 12.

26. The pharmaceutical composition of claim 25, wherein the composition is formulated as a colloidal dispersion system, macromolecular complex, nanocapsule, microsphere, bead, oil-in-water emulsion, micelle, mixed micelle, or liposome.

27. The pharmaceutical composition of any one of claims 24-26, wherein the composition is formulated for oral, intranasal, pulmonary, intradermal, transdermal, subcutaneous, intramuscular, intraperitoneal, or intravenous delivery.

28. A method of treating a patient infected with Bordetella pertussis, comprising administering to the patient the antibody of any of claims 1-16 or the pharmaceutical composition of any one of claims 24-27.

29. A method of treating a patient infected with Bordetella pertussis, comprising co-administering to the patient an effective amount of the humanized 1B7 antibody of any one of claim 1-3, 7, 9, or 10 and an effective amount of the humanized 11E6 antibody of any one of claim 4-6, 8, 11, or 12.

30. The method of claim 29, wherein the humanized 1B7 antibody and the humanized 11E6 antibody are administered simultaneously to the patient.

31. The method of claim 29, wherein the humanized 1B7 antibody is administered to the patient prior to administering the humanized 11E6 antibody to the patient.

32. The method of claim 29, wherein the humanized 1B7 antibody is administered to the patient after administering the humanized 11E6 antibody to the patient.

33. The method of claim 29, wherein co-administration of the humanized 1B7 antibody and the humanized 11E6 antibody produces synergistic effects.

34. A method of treating a patient infected with Bordetella pertussis, comprising co-administering to the patient at least one antibody of any one of claims 1-16 or the pharmaceutical composition of any one of claims 24-27, and an antimicrobial agent.

35. The method of claim 34, wherein the antimicrobial agent is selected from azithromycin, clarithromycin, erythromycin, trimethoprim-sulfamethoxasole, roxithromycin, ketolides, ampicillin, amoxicillin, tetracycline, chloramphenicol, fluoroquinolones, and cephalosporins.

36. The method of any one of claims 28-35, wherein the patient is human.

37. The method of claim 36, wherein the human is an infant.

38. A method of preventing Bordetella pertussis infection in a subject previously exposed to Bordetella pertussis, comprising administering to the subject an effective amount of the antibody of any of claims 1-16 or an effective amount of the pharmaceutical composition of any one of claims 24-27.

39. The method of any one of claims 28-38, wherein the method comprises a reduction of white blood cell count.

40. The method of any one of claims 28-39, wherein the method comprises a reduction of the duration and/or the frequency of cough.

41. The method of any one of claims 28-40, wherein the method comprises a reduction of Bordetella pertussis level in the nasopharynx and/or the lung.

42. The method of any one of claims 28-41, wherein the pertussis toxin protein is neutralized.

43. The method of claim 42, wherein the pertussis toxin protein is prevented from binding to its cellular receptor.

44. The method of claim 42, wherein the pertussis toxin protein is prevented from reaching the cellular cytosol.

45. A method of treating a patient infected with Bordetella parapertussis, comprising administering to the patient an effective amount of the antibody of any of claims 1-16 or an effective amount of the pharmaceutical composition of any one of claims 24-27.

46. A method of treating a patient infected with Bordetella parapertussis, comprising co-administering to the patient an effective amount of the humanized 1B7 antibody of any one of claim 1-3, 7, 9, or 10 and an effective amount of the humanized 11E6 antibody of any one of claim 4-6, 8, 11, or 12.

47. A method of preventing Bordetella parapertussis infection in a subject previously exposed to Bordetella pertussis, comprising administering to the subject an effective amount of the antibody of any of claims 1-16 or the pharmaceutical composition of any one of claims 24-27.


SEQUENCE LISTING (42 sequences)

SEQ ID NO: 1 - protein, 118 aa, Artificial sequence
  Artificial polypeptide
QVQLQQPGSELVRPGASVKLSCKASGYKFTSYWMHWVKQRPGQGLEWIGNIFPGSGSTNYDEKFNSKATLTVDTSSNTAYMQLSSLTSEDSAVYYCTRWLSGAYFDYWGQGTTLTVSS

SEQ ID NO: 2 - protein, 118 aa, Artificial sequence
  Artificial polypeptide
QVQLVQSGAEVKKPGASVKVSCKASGYKFTSYWMHWVRQAPGQGLEWIGNIFPGSGSTNYDEKFNSRVTLTVDTSTSTAYMELSSLRSEDTAVYYCTRWLSGAYFDYWGQGTTVTVSS

SEQ ID NO: 3 - protein, 118 aa, Artificial sequence
  Artificial polypeptide
QVQLVQSGAEVKKPGASVKVSCKASGYKFTSYWMHWVRQAPGQGLEWIGNIFPGSGSTNYAQKFQGRVTLTVDTSTSTAYMELSSLRSEDTAVYYCTRWLSGAYFDYWGQGTTVTVSS

SEQ ID NO: 4 - protein, 118 aa, Artificial sequence; identical to SEQ ID NO: 3
  Artificial polypeptide

SEQ ID NO: 5 - protein, 118 aa, Artificial sequence
  Artificial polypeptide
QVQLQQSGSELKKPGASVKISCKASGYKFTSYWMHWVKQRPGQGLEWIGNIFPGSGSTNYDEKFNSRVTLTVDTSTSTAYMELSSLRSEDTAVYYCTRWLSGAYFDYWGQGTTLTVSS

SEQ ID NO: 6 - protein, 118 aa, Artificial sequence
  Artificial polypeptide
QVQLVQSGAELVKPGASVKLSCKASGYKFTSYWMHWVKQRPGQGLEWIGNIFPGSGSTNYDEKFNSKATLTVDTSTSTAYMELSSLRSEDTAVYYCTRWLSGAYFDYWGQGTTLTVSS

SEQ ID NO: 7 - protein, 106 aa, Artificial sequence
  Artificial polypeptide
QIVLTQSPALMSASPGEKVTMTCSASSSVSFMYWYQQKPRSSPKPWIYLTSNLPSGVPARFSGSGSGTSYSLTISSMEAEDAATYYCQQWSSHPPTFGSGTKLEIK

SEQ ID NO: 8 - protein, 106 aa, Artificial sequence
  Artificial polypeptide
QIVLTQSPDFQSVTPKEKVTITCSASSSVSFMYWYQQKPDQSPKPLIYLTSNLPSGVPARFSGSGSGTSYTLTINSLEAEDAATYYCQQWSSHPPTFGSGTKVEIK

SEQ ID NO: 9 - protein, 106 aa, Artificial sequence
  Artificial polypeptide
QIVLTQSPDFQSVTPKEKVTITCRASSSVSFMYWYQQKPDQSPKPLIYLTSNLPSGVPARFSGSGSGTDYTLTINSLEAEDAATYYCQQWSSHPPTFGSGTKVEIK

SEQ ID NO: 10 - protein, 106 aa, Artificial sequence
  Artificial polypeptide
QIVLTQSPDFQSVTPKEKVTITCRASSIVSFLYWYQQKPDQSPKPLIYLASNLPSGVPARFSGSGSGTDYTLTINSLEAEDAATYYCQQWSSHPPTFGSGTKVEIK

SEQ ID NO: 11 - protein, 106 aa, Artificial sequence
  Artificial polypeptide
QIVLTQSPATLSVSPGERVTLTCSASSSVSFMYWYQQKPGRAPKPLIYLTSNLPSGVPARFSGSGSGTSYTLTINSLEAEDAATYYCQQWSSHPPTFGSGTKLEIK

SEQ ID NO: 12 - protein, 106 aa, Artificial sequence
  Artificial polypeptide
QIVLTQSPDFMSATPGEKVTMTCSASSSVSFMYWYQQKPRQSPKPWIYLTSNLPSGVPARFSGSGSGTDYTLTINSMEAEDAATYYCQQWSSHPPTFGSGTKLEIK

SEQ ID NO: 13 - protein, 123 aa, Artificial sequence
  Artificial polypeptide
EVKVVESGGGLVQPGGSLRLSCTTSGFTFTDYYVSWVRQPPGKALEWLGFIRNKVNGYTTEFSSSVKGRFTISRDNSQSILYLQMNTLRVEDSATYYCARVSYYGRGWYFDYWGQGTTLTVSS

SEQ ID NO: 14 - protein, 123 aa, Artificial sequence
  Artificial polypeptide
EVQVVESGGGLVQPGRSLRLSCTTSGFTFTDYYVSWVRQAPGKALEWLGFIRNKVNGYTTEFSSSVKGRFTISRDNSKSILYLQMNSLKIEDTAVYYCARVSYYGRGWYFDYWGQGTTVTVSS

SEQ ID NO: 15 - protein, 123 aa, Artificial sequence
  Artificial polypeptide
EVQVVESGGGLVQPGRSLRLSCTTSGFTFTDYYVSWVRQAPGKALEWVGFIRNKVNGYTTEFAASVRGRFTISRDNSKSILYLQMNSLKIEDTAVYYCARVSYYGRGWYFDYWGQGTTVTVSS

SEQ ID NO: 16 - protein, 123 aa, Artificial sequence; identical to SEQ ID NO: 15
  Artificial polypeptide

SEQ ID NO: 17 - protein, 123 aa, Artificial sequence
  Artificial polypeptide
EVQVVESGGGLVQPGGSLRLSCTTSGFTFTDYYVSWVRQPPGKALEWLGFIRNKVNGYTTEFSSSVKGRFTISRDNSKSTLYLQMNTLRVDDTAVYYCARVSYYGRGWYFDYWGQGTTLTVSS

SEQ ID NO: 18 - protein, 123 aa, Artificial sequence
  Artificial polypeptide
EVQVVESGGGLVQPGRSLRLSCTTSGFTFTDYYVSWVRQAPGKALEWLGFIRNKVNGYTTEFSSSVKGRFTISRDNSKSILYLQMNSLKIEDTAVYYCARVSYYGRGWYFDYWGQGTTLTVSS

SEQ ID NO: 19 - protein, 107 aa, Artificial sequence
  Artificial polypeptide
DIVMTQSTSSLSASLGDRVTISCRASQDIDNYLSWFQQKPDGTVKLLIYYTSRLHSGVPSRFSGSGSGTDYSLTISSLDQEDIATYFCQQGNTFPWTFGGGTKLEIK

SEQ ID NO: 20 - protein, 107 aa, Artificial sequence
  Artificial polypeptide
DIVMTQSPSSLSASVGDRVTISCRASQDIDNYLSWFQQKPGGTVKLLIYYTSRLHSGVPSRFSGSGSGTDYTLTISSLQPEDIATYFCQQGNTFPWTFGGGTKVEIK

SEQ ID NO: 21 - protein, 107 aa, Artificial sequence
  Artificial polypeptide
DIVMTQSPSSLSASVGDRVTITCRASQDIDNYLSWFQQKPGGTVKLLIYYTSRLHSGVPSRFSGSGSGTDYTLTISSLQPEDIATYFCQQGNTFPWTFGGGTKVEIK

SEQ ID NO: 22 - protein, 107 aa, Artificial sequence; identical to SEQ ID NO: 21
  Artificial polypeptide

SEQ ID NO: 23 - protein, 107 aa, Artificial sequence
  Artificial polypeptide
DIVMTQSPSSLSASVGDRVTISCRASQDIDNYLSWFQQKPGGTVKLLIYYTSRLHSGVPSRFSGSGSGTDYTLTISSLQPEDIATYFCQQGNTFPWTFGGGTKLEIK

SEQ ID NO: 24 - protein, 107 aa, Artificial sequence; identical to SEQ ID NO: 23
  Artificial polypeptide

SEQ ID NO: 25 - protein, 232 aa, Artificial sequence
  Artificial polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 10 aa, Artificial sequence
  Synthetic peptide
GYKFTSYWMH

SEQ ID NO: 27 - protein, 17 aa, Artificial sequence
  Synthetic peptide
NIFPGSGSTNYDEKFNS

SEQ ID NO: 28 - protein, 17 aa, Artificial sequence
  Synthetic peptide
NIFPGSGSTNYAQKFQG

SEQ ID NO: 29 - protein, 9 aa, Artificial sequence
  Synthetic peptide
WLSGAYFDY

SEQ ID NO: 30 - protein, 10 aa, Artificial sequence
  Synthetic peptide
SASSSVSFMY

SEQ ID NO: 31 - protein, 10 aa, Artificial sequence
  Synthetic peptide
RASSSVSFMY

SEQ ID NO: 32 - protein, 10 aa, Artificial sequence
  Synthetic peptide
RASSIVSFLY

SEQ ID NO: 33 - protein, 7 aa, Artificial sequence
  Synthetic peptide
LTSNLPS

SEQ ID NO: 34 - protein, 7 aa, Artificial sequence
  Synthetic peptide
LASNLPS

SEQ ID NO: 35 - protein, 9 aa, Artificial sequence
  Synthetic peptide
QQWSSHPPT

SEQ ID NO: 36 - protein, 10 aa, Artificial sequence
  Synthetic peptide
GFTFTDYYVS

SEQ ID NO: 37 - protein, 19 aa, Artificial sequence
  Synthetic peptide
FIRNKVNGYTTEFSSSVKG

SEQ ID NO: 38 - protein, 19 aa, Artificial sequence
  Synthetic peptide
FIRNKVNGYTTEFAASVRG

SEQ ID NO: 39 - protein, 12 aa, Artificial sequence
  Synthetic peptide
VSYYGRGWYFDY

SEQ ID NO: 40 - protein, 11 aa, Artificial sequence
  Synthetic peptide
RASQDIDNYLS

SEQ ID NO: 41 - protein, 7 aa, Artificial sequence
  Synthetic peptide
YTSRLHS

SEQ ID NO: 42 - protein, 9 aa, Artificial sequence
  Synthetic peptide
QQGNTFPWT